(19) United States
(12) Patent Application Publication
(10) Pub. No.: US 2021/0134461 A1
(43) Pub. Date: May 6, 2021

(54) METHODS AND SYSTEMS FOR PRIORITIZING COMPREHENSIVE PROGNOSES AND GENERATING AN ASSOCIATED TREATMENT INSTRUCTION SET

(71) Applicant: KPN INNOVATIONS, LLC., Lakewood, CO (US)

(72) Inventor: Kenneth Neumann, LAKEWOOD, CO (US)

(21) Appl. No.: 17/007,196

(22) Filed: Aug. 31, 2020

Related U.S. Application Data
(63) Continuation-in-part of application No. 16/668,423, filed on Oct. 30, 2019, now Pat. No. 10,847,261.

(51) Int. Cl. G16H 50/20; G16H 10/60; G16H 15/00; G16H 70/20; G16H 50/70; G16H 40/67; G16H 10/20; G16H 50/50; G06N 20/00; G06F 16/9038

(52) U.S. Cl. CPC G16H 50/20; G16H 10/60; G16H 15/00; G16H 70/20; G16H 50/70; A61B 5/6801; G16H 10/20; G16H 50/50; G06N 20/00; G06F 16/9038; G16H 40/67

(57) ABSTRACT

A system for prioritizing comprehensive prognoses and generating an associated treatment instruction set, the system comprising a computing device configured to receive at least a user biological marker and select a prognosis using a classification machine-learning model and an associated diagnostic from a biological marker database. Computing device may rank a diagnostic, wherein ranking further comprises a statistical machine-learning process to determine a figure of merit of a diagnostic for a biological marker. Computing device may use a supervised machine-learning process to select a prognosis according to a figure of merit and generate a treatment, ranking an instruction set of the treatment, and simulate the instruction set using a simulation machine-learning process to generate a prognosis, determining a rank for a prognosis, and providing the instruction set that results in the optimal prognosis. Computing device displaying, using a graphical user interface, the treatment instruction set.

### CROSS-REFERENCE TO RELATED APPLICATIONS

This application is a continuation-in-part of Non-provisional Application No. 16/668,423 filed on Oct. 30, 2019 and entitled “METHODS AND SYSTEMS FOR PRIORITIZING COMPREHENSIVE DIAGNOSES,” the entirety of which is incorporated herein by reference.

### FIELD OF THE INVENTION

The present invention generally relates to the field of machine-learning. In particular, the present invention is directed to methods and systems for prioritizing comprehensive prognoses and generating an associated treatment instruction set.

### BACKGROUND

Accurate selection and treatment of comprehensive diagnoses is imperative to achieve a vibrant constitution. Frequently, comprehensive advisors can get overwhelmed with the number of factors that are necessary to calculate and stay current on to accurately and efficiently prioritize comprehensive diagnoses. Unfortunately, many comprehensive diagnoses are left untreated due to a lack of understanding of current knowledge and guidelines.

### SUMMARY OF THE DISCLOSURE

In an aspect, a system for prioritizing comprehensive prognoses and generating an associated treatment instruction set, the system includes a computing device, wherein the computing device is designed and configured to receive at least a user biological marker, generate a classification machine-learning model using machine-learning training data wherein the machine-learning training data contains a plurality of data entries containing biological markers as inputs correlated to associated diagnostics as outputs, determine a diagnostic using the at least a user biological marker, the classification machine-learning model, and the machine-learning training data, rank the diagnostic, wherein ranking includes using a statistical machine-learning process to determine a figure of merit of the diagnostic matching the user biological marker, select a prognosis as a function of the figure of merit, wherein selecting the prognosis includes generating at least a treatment, performing a simulation machine-learning process, wherein the simulation machine-learning process generates an output containing a prognosis using the at least a treatment as an input, determining a rank for the prognosis, and providing an instruction set that results in an optimal prognosis, display, using a graphical user interface, the instruction set.

In an aspect, a method for prioritizing comprehensive prognoses and generating an associated treatment instruction set, the method including receiving, by a computing device, at least a user biological extraction, wherein the biological extraction data contains at least a biological marker of disease, generating, by the computing device, a classification machine-learning model using a machine-learning training data wherein the machine-learning training data contains a plurality of data entries containing biological markers as inputs corresponding to associated diagnostics as outputs, determining, by the computing device, a diagnostic using the at least a user biological marker, the classification machine-learning model, and the machine-learning training data, ranking, by the computing device, the diagnostic, wherein ranking includes using a statistical machine-learning process to determine a figure of merit of a diagnostic matching the user biological marker, and selecting, by the computing device, a prognosis as a function of the figure of merit, wherein selecting the prognosis includes generating at least a treatment, simulating the at least an instruction set using a simulation machine-learning process, wherein simulating using the simulation machine-learning process further comprises performing a simulation machine-learning process, wherein the simulation machine-learning process generates a prognosis that would result from implementing each instruction set, determining a rank for each prognosis, and providing, by the computing device, the instruction set that results in the optimal prognosis, and displaying, using a graphical user interface, the treatment instruction set.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for prioritizing comprehensive diagnoses;

FIG. 2 is a block diagram illustrating an exemplary embodiment of a classification module;

FIG. 3 is a block diagram illustrating an exemplary embodiment of a user database;

FIG. 4 is a block diagram illustrating an exemplary embodiment of a biological marker database;

FIG. 5A-5B are a diagrammatic representation of classification training data;

FIG. 6 is a block diagram illustrating an exemplary embodiment of a priority treatment module;

FIG. 7 is a block diagram illustrating an exemplary embodiment of a training set database;

FIG. 8 is a block diagram illustrating an exemplary embodiment of an expert database;

FIG. 9 is a diagrammatic representation of treatment training set;

FIG. 10 is a process flow diagram illustrating an exemplary embodiment of a method for prioritizing comprehensive diagnoses;

FIG. 11 is a block diagram illustrating an exemplary embodiment of a system for prioritizing comprehensive prognoses and generating an associated treatment instruction set;

FIG. 12 is a diagrammatic representation of an exemplary embodiment of a user device;

FIG. 13 is a process flow diagram illustrating an exemplary embodiment of a method for prioritizing comprehensive prognoses and generating an associated treatment instruction set; and

FIG. 14 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

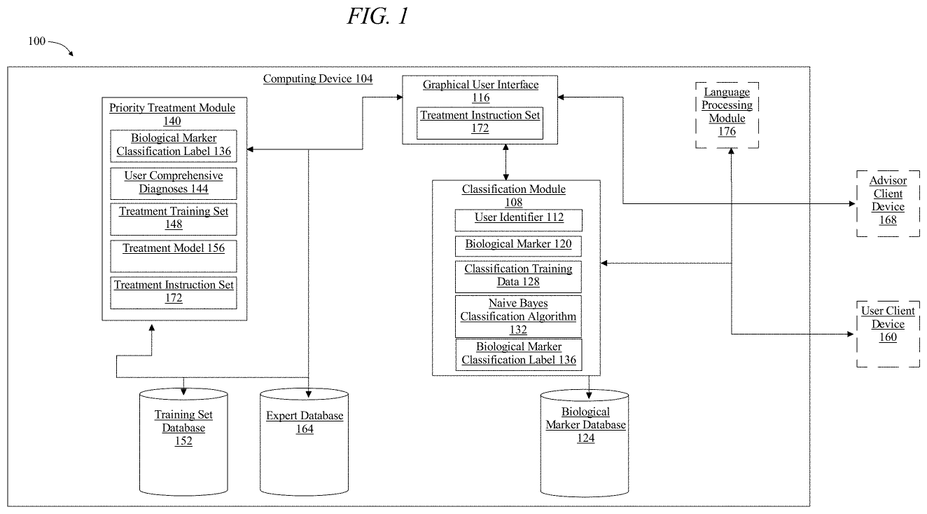

Referring now to the drawings, FIG. 1 illustrates an exemplary embodiment of a system 100 for prioritizing comprehensive diagnoses. System 100 includes a computing device 104 which may include any computing device as described herein, including without limitation a microcontroller, microcomputing device 104, digital signal computing device 104 (DSP) and/or system on a chip (SoC) as described herein. A computing device 104 may be housed with, may be incorporated in, or may incorporate one or more sensors of at least a sensor. Computing device may include, be included in, and/or communicate with a mobile device such as a mobile telephone or smartphone. A computing device 104 may include a single computing device operating independently, or may include two or more computing device operating in concert, in parallel, sequentially or the like; two or more computing devices may be included together in a single computing device or in two or more computing devices. A computing device 104 with one or more additional devices as described below in further detail via a network interface device. Network interface device may be utilized for connecting a computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. A computing device 104 may include but is not limited to, for example, A computing device 104 or cluster of computing devices in a first location and a second computing device or cluster of computing devices in a second location. A computing device 104 may include one or more computing devices dedicated to data storage, security, distribution of traffic for load balancing, and the like. A computing device 104 may distribute one or more computing tasks as described below across a plurality of computing devices of computing device, which may operate in parallel, in series, redundantly, or in any other manner used for distribution of tasks or memory between computing devices. A computing device 104 may be implemented using a “shared nothing” architecture in which data is cached at the worker, in an embodiment, this may enable scalability of system 100 and/or computing device.

With continued reference to FIG. 1, a computing device 104 may be designed and/or configured to perform any method, method step, or sequence of method steps in any embodiment described in this disclosure, in any order and with any degree of repetition. For instance, a computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device 104 may perform any step or sequence of steps as described in this disclosure in parallel, such as simultaneously and/or substantially simultaneously performing a step two or more times using two or more parallel threads, computing device 104 cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, system 100 includes a classification module 108 which may be implemented as any hardware and/or software module. Classification module 108 is designed and configured to receive a user identifier 142 entered by a comprehensive advisor located on a graphical user interface operating on a processor, retrieve a user biological marker 120 from a user database, receive classification training data wherein the classification training data contains a plurality of data entries including biological marker data containing alert and non-alert labels and generate a naïve Bayes classification algorithm utilizing classification training data wherein the naïve Bayes classification algorithm utilizes the user biological marker as an input and outputs a biological marker classification label.

With continued reference to FIG. 1, a “user identifier” as used in this disclosure, includes any data that uniquely identifies a particular user. Data may include a user's name, a user's date of birth, a user's medical identification number, a public and/or private key pair, a cryptographic hash, a biometric identifier such as an iris scan, fingerprint scan, a palm vein scan, a retina scan, facial recognition, DNA, a personal identification number, a driver's license or passport, token-based identification systems, digital signatures, and the like. Uniqueness may include uniqueness within system 100 such as ensuring that a particular user identifier is not already utilized by another user. Uniqueness may include a statistically ensured uniqueness such as a global unique identifier (GUID), or a unique identifier identification (UID).

With continued reference to FIG. 1, a user identifier 142 is entered by a comprehensive advisor. A “comprehensive advisor” as used in this disclosure, includes a person who is licensed by a state and/or federal licensing agency that may help in identifying, preventing, and/or treating illness and/or disability. A comprehensive advisor may include persons such as a functional medicine doctor, a doctor of osteopathy, a nurse practitioner, a physician assistant, a Doctor of Optometry, a doctor of dental medicine, a doctor of dental surgery, a naturopathic doctor, a doctor of physical therapy, a nurse, a doctor of chiropractic medicine, a doctor of oriental medicine and the like. A comprehensive advisor may include other skilled professionals such as nurses, respiratory therapists, pharmacists, home health aides, audiologists, clinical nurse specialists, nutritionists, dieticians, clinical psychologists, psychiatric mental health nurse practitioners, spiritual coaches, life coaches, holistic medicine specialists, acupuncturists, reiki masters, yoga instructors, holistic health coaches, wellness advisors and the like.

With continued reference to FIG. 1, a user identifier 142 is entered by a comprehensive advisor on a graphical user interface 146 operating on the processor. Graphical user interface may interact with a remote device such as a device in communication with system 1000 such as user client device and/or advisor client device through hypertext markup language (HTML) to be displayed on a remote device. In an embodiment, graphical user interface 146 may be displayed on a remote device as a web form. Graphical user interface 146 may include without limitation, a form or other graphical element having data entry fields, where a comprehensive advisor may enter a user identifier 142. Graphical user interface 146 may include data entry fields that allow for a comprehensive advisor to enter free form textual inputs. Graphical user interface 146 may provide drop-down lists, where users such as comprehensive advisors may be able to select one or more entries to indicate one or more users. Graphical user interface 146 may include touch options where a user may enter a command by touching and selecting a particular option. Graphical user interface 146 may include text to speech software whereby a comprehensive advisor may speak a particular command such as a user identifier 142 and graphical user interface 146 may convert the spoken command into a textual output that is displayed on graphical user interface 146.

With continued reference to FIG. 1, a “biological marker” as used in this disclosure, includes a physically extracted sample, which as used herein, includes a sample obtained by removing and analyzing tissue and/or fluid. Physically extracted sample may include without limitation a blood sample, a tissue sample, a buccal swab, a mucous sample, a stool sample, a hair sample, a fingernail sample, genetic sample, or the like. At least a genetic sample may include a complete genome of a person or any portion thereof. At least a genetic sample may include a DNA sample and/or an RNA sample. Biological marker 120 may include an epigenetic sample, a proteomic sample, a tissue sample, a biopsy, and/or any other physically extracted sample such as an endocrinal sample.

In further non-limiting examples, the at least a biological marker 120 may include a signal from at least a sensor configured to detect physiological data of a user and recording the at least a biological marker 120 as a function of the signal, for instance as from a wearable device. At least a sensor may include any medical sensor and/or medical device configured to capture sensor data concerning a user, including any scanning, radiological and/or imaging device such as without limitation x-ray equipment, computer assisted tomography (CAT) scan equipment, positron emission tomography (PET) scan equipment, any form of magnetic resonance imagery (MRI) equipment, ultrasound equipment, optical scanning equipment such as photo-plethysmographic equipment, electromagnetic sensor, such as an electroencephalographic sensor, magnetoencephalographic sensor, electrocardiographic sensor, and electromyographic sensor, and/or temperature sensor. At least a sensor may include any sensor that may be included in a mobile device and/or wearable device, including without limitation a motion sensor such as an inertial measurement unit (IMU), one or more accelerometers, one or more gyroscopes, one or more magnetometers, or the like. At least a wearable and/or mobile device sensor may capture step, gait, and/or other mobility data, as well as data describing activity levels and/or physical fitness. In non-limiting illustrative examples, at least a wearable and/or mobile device sensor may detect heart rate, a hematological parameter including blood oxygen level, pulse rate, heart rate, pulse rhythm, blood sugar, and/or blood pressure, external biomarkers and/or readings. At least a sensor may be a part of system 100 or may be a separate device in communication with system 100, as described in further detail below

Still referring to FIG. 1, at least a biological marker 120 may include any result of any medical test, physiological assessment, cognitive assessment, psychological assessment, or the like. System 100 may receive at least a biological marker 120 from one or more other devices; system 100 may alternatively or additionally perform one or more assessments and/or tests to obtain at least a biological marker 120, and/or one or more portions thereof, on system 100. For instance, at least biological marker 120 may include or more entries by a user in a form or similar graphical user interface 146 object; one or more entries may include, without limitation, user responses to questions on a psychological, behavioral, personality, or cognitive test. For instance, a processor may present to user a set of assessment questions designed or intended to evaluate a current state of mind of the user, a current psychological state of the user, a personality trait of the user, or the like; a processor may provide user-entered responses to such questions directly as at least a biological marker 120 and/or may perform one or more calculations or other algorithms to derive a score or other result of an assessment as specified by one or more testing protocols, such as automated calculation of a Stanford-Binet and/or Wechsler scale for IQ testing, a personality test scoring such as a Myers-Briggs test protocol, or other assessments that may occur to persons skilled in the art upon reviewing the entirety of this disclosure.

With continued reference to FIG. 1, at least a biological marker 120 may include assessment and/or self-assessment data, and/or automated or other assessment results, obtained from a third-party device; third-party device may include, without limitation, a server or other device (not shown) that performs automated cognitive, psychological, behavioral, personality, or other assessments. Third-party device may include a device operated by an informed advisor.

Still referring to FIG. 1, at least a biological marker 120 may include data describing one or more test results, including results of mobility tests, stress tests, dexterity tests, endocrinal tests, genetic tests, and/or electromyographic tests, biopsies, radiological tests, genetic tests, and/or sensor tests. Persons skilled in the art, upon reviewing the entirety of this disclosure, will be aware of various additional examples of at least a physiological sample consistent with this disclosure.

With continued reference to FIG. 1, a user biological marker 120 may be stored in a user biological marker database 124. Biological marker includes any of the biological markers as described above. Biological marker database 124 may be implemented, without limitation, as a relational database, a key-value retrieval datastore such as a NOSQL database, or any other format or structure for use as a datastore that a person skilled in the art would recognize as suitable upon review of the entirety of this disclosure. Biological marker database 124 may include one or more data entries containing one or more user biological marker 120. Processor may select one or more user biological marker 120 from biological marker database 124 by evaluating a user identifier 142 entered by a comprehensive advisor on a graphical user interface 146 to a user identifier 142 located within biological marker database 124. In an embodiment, each biological marker 120 contained within biological marker database 124 may contain an individual user identifier 142. A processor may evaluate biological marker 120 by comparing user identifier 142 to determine if they are identical and belong to the same user. For instance and without limitation, a processor may evaluate a user identifier 142 generated by a comprehensive advisor on a graphical user interface 146 that includes a user's name and date of birth to a user identifier 142 contained within biological marker database 124 that includes a user's name and date of birth. In such an instance, processor may evaluate both usernames and dates of birth to determine if they are identical and match. Evaluating may include other techniques such as comparing hash values and authenticating public and private key pairs. Processor may continue to retrieve a user biological marker 120 from biological marker database 124 after confirming that user identifier 142 match. In an embodiment, processor may not proceed to retrieve a user biological marker 120 from a biological marker database 124 when user identifier 142 do not match.

With continued reference to FIG. 1, classification module 108 is configured to receive classification training data 128. “Training data,” as used in this disclosure, is data containing correlation that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by at least a server may correlate any input data as described in this disclosure to any output data as described in this disclosure.

With continued reference to FIG. 1, classification training data 128 includes a plurality of data entries including biological marker 120 data containing alert and non-alert classification labels. Biological marker 120 data includes any of the biological marker 120 data as described above. A “classification label” as used in this disclosure, includes an indicator that a particular data entry belongs to a specific class based on a common property and/or attribute. Classification training data 128 contains observations whose category membership or classification labels are already known. Classification labels are generated by classification algorithms. Classification algorithms may include generating classifications models that draws a conclusion from input values given for training data. Classification algorithms predict classification labels for new data. Classification training data 128 includes biological marker 120 data containing alert and non-alert classification labels. An “alert classification label” as used in this disclosure, includes an indicator that a particular biological marker 120 represents an alert condition. An alert condition includes an instance where a particular biological marker 120 is outside of normal reference ranges, indicates a potentially life threatening condition, indicates abnormal findings, indicates the need for immediate medical attention, and the like. For instance and without limitation, a biological marker 120 such as a serum sodium level of 155 mEq/L may be classified to contain an alert classification label when compared to a reference range of serum sodium levels between 135 and 145 mEq/L. A “non-alert classification label” as used in this disclosure, includes an indicator that a particular biological marker 120 does not contain an alert condition. A non-alert condition includes an instance where a particular biological marker 120 is within normal reference ranges, does not indicate a potentially life-threatening condition, indicates normal findings, does not indicate the need for immediate medical attention and the like. For instance and without limitation, a biological marker 120 such as an electroencephalography (EEG) that contains no abnormal findings may be classified to contain a non-alert classification label.

With continued reference to FIG. 1, classification module 108 is configured to generate a naïve Bayes classification algorithm 132. Naïve Bayes classification algorithm generates classifiers by assigning class labels to problem instances, represented as vectors of feature values. Class labels are drawn from a finite set. Naïve Bayes classification algorithm may include generating a family of algorithms that assume that the value of a particular feature is independent of the value of any other feature, given a class variable. Naïve Bayes classification algorithm may be based on Bayes Theorem expressed as P(A/B)=P(B/A) P(A)÷(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming classification training data 128 into a frequency table. Classification module 108 may then calculate a likelihood table by calculating probabilities of different data entries and classification labels. Classification module 108 utilizes a naïve Bayes equation to calculate a posterior probability for each class. A class containing the highest posterior probability is the outcome of prediction. Naïve Bayes classification algorithm 132 may include a gaussian model that follows a normal distribution. Naïve Bayes classification algorithm may include a multinomial model that is used for discrete counts. Naïve Bayes classification algorithm 132 may include a Bernoulli model that may be utilized when feature vectors are binary. Naïve Bayes classification algorithm 132 utilizes classification training data 128 and a user biological marker 120 as an input to output a biological marker classification label 136. A “biological marker classification label” as used in this disclosure, includes a classification label generated for a user biological marker 120. A classification label includes an identifier that indicates whether a particular biological marker 120 belongs to a particular class or not. In an embodiment, a biological marker classification label 136 may include an alert or a non-alert classification label. In an embodiment, a biological marker classification label 136 may include other classification labels such as classification labels that may indicate progression of a particular disease, progression of a particular treatment and the like.

With continued reference to FIG. 1, classification module 108 may be configured to perform classification utilizing one or more other classification algorithms. Classification algorithms include but are not limited to logistic regression, fisher's linear discriminant, least squares support vector machines, quadratic classifiers, k-nearest neighbor, support vector machines, decision trees, boosted trees, random forest, neural networks, learning vector quantization and the like.

With continued reference to FIG. 1, system 100 includes a priority treatment module 140 which may be implemented as any hardware and/or software module. Priority treatment module 140 is designed and configured to receive the biological marker classification label 136 from the classification module 108; receive a plurality of user comprehensive diagnoses 144 generated by a comprehensive advisor on a graphical user interface 146 operating on the processor; select a treatment training set 148 as a function of the biological marker classification label 136 wherein the treatment training set 148 correlates comprehensive diagnoses to prioritized treatment facets; generate using a supervised machine-learning model a treatment 156 that outputs an ordered priority treatment list for each of the plurality of comprehensive diagnoses utilizing the selected treatment training set; evaluate the priority treatment for each of the plurality of comprehensive diagnoses; generate a treatment instruction set 172 wherein the treatment instruction set 172 further comprises generating an ordered treatment plan for the plurality of comprehensive diagnoses; and display the ordered treatment instruction set 172 on a graphical user interface 146 located on the processor.

With continued reference to FIG. 1, priority treatment module 140 is configured to receive a plurality of user comprehensive diagnoses 144 generated by a comprehensive advisor on a graphical user interface 146 operating on a processor. A “comprehensive diagnosis” as used in this disclosure, includes a disease and/or condition diagnosed by a comprehensive advisor. A comprehensive diagnosis may explain particular signs and symptoms experienced by a user. A comprehensive diagnosis may be generated from information gathered from a medical history, physical examination, one or more diagnostic procedures such as medical tests, and one or more biological marker 120. For instance and without limitation, a comprehensive advisor such as a functional medicine doctor may diagnose a user with a comprehensive diagnosis of lupus after user complains of symptoms that include achy joints, unexplained fever, skin rash, and a butterfly shaped rash across user's cheeks and nose. In such an instance, comprehensive advisor may base a lupus diagnosis on user's symptoms and on medical tests such as blood and urine tests, antinuclear antibody (ANA) tests and the like. A “plurality of user comprehensive diagnoses” as used in this disclosure, includes one or more comprehensive diagnoses pertaining to a particular user. For example, a plurality of user comprehensive diagnoses may include estrogen dominance, back spasm, tinnitus, and sinus infection all of which may pertain to the same user and all of which may have been diagnosed by one or more comprehensive advisors.

A “treatment,” as used herein is any medication, pharmaceutical, surgery, medical intervention, prosthetic, diet, exercise, user action, lifestyle change, and/or any chemical, biological, and/or physiological adaptation prescribed and/or proscribed to a user. In non-limiting illustrative examples, a treatment may be a course of action prescribed to a user in addressing a symptom, diagnosis, condition, or the like, that can result in a prognosis, wherein the prognosis is a determined outcome from using the treatment 156, as described in further detail below.

With continued reference to FIG. 1, a plurality of user comprehensive diagnoses 144 are entered by a comprehensive advisor on a graphical user interface 146 operating on a processor. Graphical user interface 146 may include any of the graphical user interface 146 as described above. In an embodiment, a comprehensive advisor may enter into a free form text box one or more comprehensive diagnoses. In an embodiment, comprehensive advisor may enter a user identifier 142 whereby a list of comprehensive diagnoses associated with a user may be generated and displayed on a graphical user interface 146 so that comprehensive advisor may select one or more comprehensive diagnoses. In an embodiment, one or more comprehensive diagnoses may be retrieved from user database as described in more detail below.

With continued reference to FIG. 1, system 100 may include user database. User database may be implemented as any data structure suitable for use as biological marker database 124 as described above. User database may include one or more data entries containing information relevant to a particular user. For instance and without limitation, user database may include one or more comprehensive diagnoses. In yet another non-limiting example, user database may include one or more meeting summaries from an appointment with a comprehensive advisor. User database is described in more detail below.

With continued reference to FIG. 1, priority treatment module 140 selects a treatment training set 148 as a function of a biological marker classification label 136. Treatment training set includes any of the training data as described above. Treatment training set contains a plurality of data entries containing comprehensive diagnoses correlated to one or more prioritized treatment facets. “Prioritized treatment facet” as used in this disclosure, includes a treatment plan for a particular diagnosis that indicates what aspect of the particular diagnosis needs to be treated first, what needs to be treated next and what needs to be addressed in a stepwise approach. Prioritized may include a particular order of steps which need to be completed in a particular order. An aspect of a particular diagnosis includes one or more body systems, one or more body parts, one or more cellular processes, one or more complications and the like that may be impacted by the particular diagnosis. For instance and without limitation, a particular diagnosis such as Type 2 Diabetes Mellitus may affect body parts such as eyes, kidneys, nerves, heart, blood vessels, gums, feet, skin, and liver, where each body part may need to be addressed and treated at a particular time. In yet another non-limiting example, a particular diagnosis such as Lyme Disease may affect one or more body systems including the nervous system, the genitourinary system, the cardiovascular system, the immune system, joints, skin, endocrine system and the like. A priority treatment for Lyme Disease may include a treatment plan that focusses first on eradicating bacteria, then supporting the immune system, then tissue support of the joints, then fixing endocrinal imbalances such as balancing hormone levels, and finally symptomatic control. Treatment facets may be prioritized, where they may be generated in a stepwise approach that indicates which facet needs to be addressed and treated first, which facet needs to be treated second, and the like.

With continued reference to FIG. 1, treatment training set 148 may include a plurality of data entries containing comprehensive diagnoses correlated to priority treatment facets. For instance and without limitation, a treatment training set 148 may include a plurality of data entries containing comprehensive diagnoses that include polycystic ovarian syndrome (PCOS) correlated to priority treatment that includes a treatment plan consisting of first addressing dietary issues, second improving intestinal health, third addressing spiritual aspects of health, fourth addressing activity and fitness regimens, and fifth addressing nutraceuticals and supplementation. In yet another non-limiting example, treatment training set 148 may include a plurality of data entries containing comprehensive diagnoses that include hypothyroidism correlated to priority treatment that includes a treatment plan consisting of first detecting and correcting nutrient deficiencies, second decreasing stress through meditative techniques, support groups or psychotherapy, third improving diet by initiating an anti-inflammatory diet low in carbohydrates, fourth initiating an exercise routine, fifth utilizing chelating therapy to remove heavy metals, sixth eliminating offending medications, and seventh detoxifying any and all offending toxins inside of the body.

With continued reference to FIG. 1, treatment training set 148 may be stored within a training set database 152. Training set database 152 may include any data structure suitable for use as biological marker database 124 as described above in more detail. Priority treatment module 140 selects a treatment training set 148 as a function of a biological marker classification label 136. In an embodiment, treatment training set 148 contained within training set database 152 may be organized by diagnosis and/or biological marker classification label 136. Treatment training set 148 may contain classifier labels that may indicate particular diagnoses and/or biological marker 120 contained within a particular treatment training set 148. A “classifier label” as used in this disclosure, includes any category for data including a predication generated from a classification algorithm. Classification algorithms may include for example, logistic regression, least squares support vector machines, quadratic classifiers, kernel estimation, k-nearest neighbor, decision trees, random forests, neural networks, and/or learning vector quantization. A processor may select a treatment training set 148 containing a classifier label that matches a biological marker classification label 136. For instance and without limitation, a processor may match a treatment training set 148 containing an alert classifier label to a biological marker classification label 136 that contains an alert label. In yet another non-limiting example, a processor may match a treatment training set 148 containing a non-alert classifier label to a biological marker classification label 136 that contains a non-alert classifier label.

With continued reference to FIG. 1, priority treatment module 140 may select a treatment training set 148 by classifying user comprehensive diagnoses 144 to generate comprehensive diagnosis classification labels. Priority treatment module 140 may receive diagnostic training data, where diagnostic training data contains a plurality of data entries containing urgent and non-urgent labels. Diagnostic training data may include any of the training data as described above. An “urgent” classification label as used in this disclosure, indicates a diagnosis that requires immediate medical attention. Immediate medical attention may include for example, medical attention that is warranted within the immediate 1-2 weeks. For instance and without limitation, a diagnosis such as chest pains due to myocardial infarction, slurred speech, head injury, concussion, broken bones, influenza, seizures, serious burns, poisonings, deep gunshot wounds and the like may be diagnoses that require urgent medical attention. A “non-urgent” classification label as used in this disclosure, indicates a diagnosis that does not require immediate medical attention and can either be treated at a later point in time or may not require medical attention at all and rather may be treated by a user at home using self-care techniques. For instance and without limitation, a diagnosis such as hammertoe, pulled muscle, sprained finger, Alzheimer's disease, persistent cough, rhinovirus, minor laceration, ocular pruritus, skin rash, and the like. Priority treatment module 140 generates a classification algorithm utilizing diagnostic training data. Classification algorithm may include any classification algorithm including for example, logistic regression, fisher's linear discriminant, least squares support vector machines, quadratic classifiers, k-nearest neighbor, support vector machines, decision trees, boosted trees, random forest, neural networks, learning vector quantization, and/or any classification algorithm as described in this disclosure. Classification algorithm utilizes a plurality of user comprehensive diagnoses 144 as inputs and outputs a comprehensive diagnosis classification label for each of the plurality of user comprehensive diagnoses 144. For instance and without limitation, a first user diagnosis such as tension headache may contain a non-urgent classification label while a second user diagnosis such as myocardial infarction may include an urgent classification label. Priority treatment module 140 may select a treatment training set 148 as a function of a comprehensive diagnosis classification label for each of the plurality of user comprehensive diagnoses 144. For example, priority treatment module 140 may select a treatment training set 148 by matching a comprehensive diagnosis classification label to a classification label contained within training set database 152. In an embodiment, priority treatment module 140 may select a treatment training set 148 by matching a comprehensive diagnosis to a comprehensive diagnosis contained within a particular treatment training set 148. For instance and without limitation, a comprehensive diagnosis generated by a comprehensive advisor may include a diagnosis of Parkinson's disease. In such an instance, priority treatment module 140 may select a treatment training set 148 containing one or more data entries containing a comprehensive diagnosis of Parkinson's disease correlated to priority treatment. In yet another non-limiting example, a comprehensive diagnosis generated by a comprehensive advisor may include a diagnosis of herpes zoster, whereby priority treatment module 140 may select a treatment training set 148 containing one or more data entries containing a comprehensive diagnosis of herpes zoster correlated to priority treatment.

With continued reference to FIG. 1, priority treatment module 140 is configured to generate using a supervised machine-learning algorithm a treatment 156 that outputs an ordered priority treatment list for each of the plurality of comprehensive diagnoses utilizing the selected treatment training set. Supervised machine-learning algorithms, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may use elements of comprehensive diagnoses as inputs, priority treatments as outputs, and a scoring function representing a desired form of relationship to be detected between elements of comprehensive diagnoses and priority treatments; scoring function may, for instance, seek to maximize the probability that a given element of a comprehensive diagnosis is associated with a given priority treatment and/or combination of comprehensive diagnoses to minimize the probability that a given element of a comprehensive diagnosis and/or combination of elements comprehensive diagnoses are not associated with a given priority treatment and/or combination of priority treatments. Scoring function may be expressed as a risk function representing an “expected loss” of an algorithm relating inputs to outputs, where loss is computed as an error function representing a degree to which a prediction generated by the relation is incorrect when compared to a given input-output pair provided in a training set. Persons skilled in the art, upon reviewing the entirety of this disclosure, will be aware of various possible variations of supervised machine-learning algorithms that may be used to determine relation between comprehensive diagnoses and priority treatments. In an embodiment, one or more supervised machine-learning algorithms may be restricted to a particular domain for instance, a supervised machine-learning process may be performed with respect to a given set of parameters and/or categories of parameters that have been suspected to be related to a given set of comprehensive diagnoses, and/or are specified as linked to a medical specialty and/or field of medicine covering a particular body system or medical specialty. As a non-limiting example, a particular set of diagnoses that indicate emergency medical conditions may be typically associated with a known urgency to seek medical attention and be treated, and a supervised machine-learning process may be performed to relate those comprehensive diagnoses to priority treatments; in an embodiment, domain restrictions of supervised machine-learning procedures may improve accuracy of resulting models by ignoring artifacts in training data. Domain restrictions may be suggested by experts and/or deduced from known purposes for particular evaluations and/or known tests used to evaluate priority treatments. Additional supervised learning processes may be performed without domain restrictions to detect, for instance, previously unknown and/or unsuspected relationships between comprehensive diagnoses and priority treatments.

With continued reference to FIG. 1, treatment 156 is a machine-learning process that may include linear or polynomial regression algorithms, may include calculating one or more equations, may include a set of instructions to generate outputs based on inputs which may be derived using any machine-learning algorithm and the like.

With continued reference to FIG. 1, priority treatment module 140 evaluates priority treatment facets for each of the plurality of comprehensive diagnoses. Evaluating priority treatment may include evaluating each of the stepwise approaches contained within a treatment plan to find overlap and eliminate competing facets. For instance and without limitation, evaluating priority treatment facets may include evaluating a stepwise approach contained within a priority treatment for a first comprehensive diagnosis such as tension headache which includes a first facet approach to correct nutritional deficiencies, a second facet approach to institute a meditation practice, and a third facet approach to initiate an anti-inflammatory diet. Priority treatment module 140 may evaluate priority treatment facets for a first comprehensive diagnosis in conjunction with a second comprehensive diagnosis such as persistent depressive disorder which includes a first facet approach to initiate an anti-inflammatory diet, a second facet approach which includes initiating an exercise regimen, a third facet approach which includes addressing gastrointestinal disbalances, and a fourth facet approach which includes initiating an anti-depressant medication. Evaluating may include comparing priority treatments to identify shared facet approaches. For instance in the above example, first comprehensive diagnosis includes a facet approach to initiate an anti-inflammatory diet and second comprehensive diagnosis includes a facet approach to initiate an anti-inflammatory diet. In yet another non-limiting example, evaluating may include identifying facets that need to be completed first before other facets can be addressed. For instance and without limitation, in the above example priority treatment module 140 may identify that correcting nutritional deficiencies needs to be performed before gastrointestinal disbalances can be addressed.

With continued reference to FIG. 1, evaluating priority treatment includes retrieving an element of user symptom data from user database. An “element of user symptom data” as used in this disclosure, includes an element of data describing one or more symptoms that a user may be experiencing currently, one or more symptoms that a user may have experienced in the past, and/or one or more symptoms that a user may be experiencing recurrently or intermittently. In an embodiment, an element of user symptom data may be received from a user client device 160. User client device 160 may include without limitation, a display in communication with a processor, where a display may include any display as described herein. User client device 160 may include an additional computing device, such as a mobile device, laptop, desktop computer and the like. With continued reference to FIG. 1, priority treatment module 140 correlates an element of user symptom data to a comprehensive diagnosis. Correlating, as used herein, may include any relation established therein linking an element of user symptom data to a comprehensive diagnosis. Correlation may include a relation established where a particular element of user symptom data is attributed to and/or caused by a comprehensive diagnosis. For instance and without limitation, an element of user symptom data such as a runny nose may be correlated to a comprehensive diagnosis such as rhinovirus. In yet another non-limiting example, an element of user symptom data such as uncontrollable jerking movements of the arms and legs may be correlated to a comprehensive diagnosis such as epilepsy. In an embodiment, priority treatment module 140 may receive a plurality of comprehensive diagnoses and correlate a plurality of elements of user symptom data to one or more diagnoses. For instance and without limitation, priority treatment module 140 may receive a plurality of comprehensive diagnoses that include hypertension, pulmonary edema, schizophrenia, and heavy metal toxicity in addition to a plurality of elements of user symptom data that include throbbing headache, fatigue, and nighttime wakening. In such an instance, priority treatment module 140 may correlate throbbing headache to hypertension, fatigue to heavy metal toxicity, and nighttime wakening to schizophrenia. In an embodiment, one or more elements of user symptom data may be correlated to one or more comprehensive diagnoses. In an embodiment, one or more comprehensive diagnoses may be correlated to one or more elements of user symptom data. Priority treatment module 140 may correlate elements of user symptom data to one or more diagnoses using learned associations. In an embodiment, priority treatment module 140 may receive correlation training data that may include a plurality of data entries containing an element of symptom data correlated to a comprehensive diagnosis. Correlation training data may include any training data as described herein. For instance and without limitation, correlation training data may include a plurality of data entries containing symptom data such as slurred speech to a comprehensive diagnosis such as stroke. Priority treatment module 140 may correlate elements of user symptom data to one or more diagnoses by consulting an expert database 164 that may contain expert input regarding one or more elements of symptom data correlated to one or more comprehensive diagnoses. Expert database 164 may include any data structure suitable for use as biological marker database 124 as described above. Expert database 164 may include one or more data entries generated by top experts in a particular field of knowledge, expert scientific articles, journals, literature, and the like as described in more detail below. User symptom data correlated to one or more comprehensive diagnoses may be utilized to generate a treatment instruction set 172 as described below in more detail. Expert database 164 may receive inputs from advisor client device 168. Advisor client device 168 may include any device suitable for use as user client device 160 as described above.

With continued reference to FIG. 1, priority treatment module 140 is configured to generate a treatment instruction set 172. A “treatment instruction set” as used in this disclosure, includes a stepwise recommended approach for the treatment by a comprehensive advisor of one or more comprehensive diagnoses. Treatment instruction set may include a series of one or more textual statements. Treatment instruction set may be altered and/or transformed by priority treatment module 140 to be able to be displayed on a graphical user interface. For example, treatment instruction set containing a series of one or more textual statements may be transformed by priority treatment module 140 to be displayed on a graphical user interface to be displayed by converting one or more entries into characters or numerical outputs readable by a computing device 104. A stepwise recommended approach includes a series of one or more facets of each comprehensive diagnosis that must be addressed and treated first before another facet can be addressed. A stepwise recommended approach includes facets from one or more comprehensive diagnoses. In an embodiment, priority treatment module 140 may evaluate one or more facets from one or more comprehensive treatments to generate a treatment instruction set 172 that includes a stepwise recommended approach for all of the plurality of user comprehensive diagnoses 144 put together. Generating a treatment instruction set 172 may include receiving input from one or more comprehensive advisors. Priority treatment module 140 may receive a comprehensive input descriptor generated by a comprehensive advisor on a graphical user interface 146 located on a processor. A “comprehensive input descriptor” as used in this disclosure, includes a summary detailing one or more encounters between a comprehensive advisor and a user. An “encounter” as used in this disclosure, includes one or more consultation events between a comprehensive advisor and a user. A consultation event may include an in person face to face meeting, a telephonic meeting, a telegraphic meeting such as a meeting conducted over a network interface, an email communication, a communication processed over a messaging service and the like. A comprehensive input descriptor may contain one or more advisory interaction summaries containing information detailing what a comprehensive advisor's own treatment plan is for a user or what the comprehensive advisor wishes to focus on next. An advisory interaction summary may contain a description of one or more comprehensive diagnoses pertaining to a user such as how advanced a particular comprehensive diagnosis is or what stage a particular comprehensive diagnosis is currently at. An advisory interaction summary may contain a description of one or more findings a comprehensive advisor discovered during a physical examination of a user. An advisory interaction summary may contain a description of one or more symptoms a user may have discussed with comprehensive advisor during an encounter. Comprehensive input descriptor may be utilized to generate an ordered treatment plan. In an embodiment, an advisory interaction summary that contains a description of one or more symptoms that a user is currently experiencing may cause one or more prioritized treatment facets to be placed ahead of another treatment facet or for one treatment facet to be placed behind another treatment facet when generating ordered treatment plan. For instance and without limitation, an advisory interaction summary that contains a description of a symptom a user is experiencing such as toe pain as a result of a comprehensive diagnosis of gout, may be utilized to place a first treatment facet that contains a diet low in uric acid ahead of a second treatment facet that contains a recommendation to initiate heavy metal detoxification. In yet another non-limiting example, an advisory interaction summary that contains a description from a physical examination of a user may contain a description of a current finding on a user's body that contains a description of multiple petechiae on the skin. In such an instance, generating an ordered treatment plan may include placing a first treatment facet that contains initiating an anti-inflammatory diet behind a second treatment facet that contains directions to apply hydrocortisone cream to the body area affected by petechiae, when generating an ordered treatment plan.

With continued reference to FIG. 1, data describing an advisory interaction summary and/or data describing one or more expert inputs may alternatively or additionally be extracting from one or more documents or entries utilizing a language processing module 176. Language processing module 176 may include any hardware and/or software module. Language processing module 176 may be configured to extract, from the one or more documents, one or more words. One or more words may include, without limitation, strings of one or characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring to FIG. 1, language processing module 176 may compare extracted words to categories of advisory inputs, such data for comparison may be entered on computing device 104 as described above using expert data inputs or the like. In an embodiment, one or more categories may be enumerated, to find total count of mentions in such documents. Alternatively or additionally, language processing module 176 may operate to produce a language processing model. Language processing model may include a program automatically generated by computing device 104 and/or language processing module 176 to produce associations between one or more words extracted from at least a document and detect associations, including without limitation mathematical associations, between such words, and/or associations of extracted words with categories of dietary data, relationships of such categories to alimentary labels, and/or categories of alimentary labels. Associations between language elements, where language elements include for purposes herein extracted words, categories of advisory interactions, relationships of such categories to users, and/or categories of expert inputs may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of advisory interaction summary, a given relationship of such categories to users, and/or a given category of expert inputs. As a further example, statistical correlations and/or mathematical associations may include probabilistic formulas or relationships indicating a positive and/or negative association between at least an extracted word and/or a given category of advisory interaction summaries, a given relationship of such categories to users, and/or a given category of expert inputs; positive or negative indication may include an indication that a given document is or is not indicating a category of an advisory interaction summary, relationship of such category to a user, and/or category of expert inputs is or is not significant. For instance, and without limitation, a negative indication may be determined from a phrase such as “joint pain was not found to be associated with hypothyroidism” whereas a positive indication may be determined from a phrase such as “joint pain was found to be associated with osteoarthritis” as an illustrative example; whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device 104, or the like.

Still referring to FIG. 1, language processing module 176 and/or computing device 104 may generate the language processing model by any suitable method, including without limitation a natural language processing classification algorithm; language processing model may include a natural language process classification model that enumerates and/or derives statistical relationships between input term and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. in an alternative or additional approach, sequential tokens may be modeled as chains, serving as the observations in a Hidden Markov Model (HMM). HMMs as used herein, are statistical models with inference algorithms that that may be applied to the models. In such models, a hidden state to be estimated may include an association between an extracted word and a category of an advisory interaction summary, a given relationship of such categories to users, and/or a given category of expert inputs. There may be a finite number of category of dietary data, a given relationship of such categories to advisory interaction summaries, and/or a given category of expert input to which an extracted word may pertain; an HMM inference algorithm, such as the forward-backward algorithm or the Viterbi algorithm, may be used to estimate the most likely discrete state given a word or sequence of words. Language processing module 176 may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 1, language processing module 176 may use a corpus of documents to generate associations between language elements in a language processing module 176, and computing device 104 may then use such associations to analyze words extracted from one or more documents and determine that the one or more documents indicate significance of a category of advisory interaction summary, a given relationship of such categories to users, and/or a given category of expert inputs. In an embodiment, computing device 104 may perform this analysis using a selected set of significant documents, such as documents identified by one or more experts as representing good science, good clinical analysis, or the like; experts may identify or enter such documents via graphical user interface 146 as described below in more detail or may communicate identities of significant documents according to any other suitable method of electronic communication, or by providing such identity to other persons who may enter such identifications into computing device 104. Documents may be entered into computing device 104 by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, computing device 104 may automatically obtain the document using such an identifier, for instance by submitting a request to a database or compendium of documents such as JSTOR as provided by Ithaka Harbors, Inc. of New York.

With continued reference to FIG. 1, generating treatment instruction set 172 includes retrieving a user diagnostic factor input from user database where the user diagnostic factor input includes a long-term target indicator and a short-term target indicator. A “user diagnostic factor input” as used in this disclosure, includes any data describing a user's health goals as they relate to a particular comprehensive diagnosis. “User health goals” as used in this disclosure, includes any user desire or outcome a user seeks to achieve in reference to a particular comprehensive diagnosis. A user diagnostic factor input includes a long-term target indicator. A “long term target indicator” as used in this disclosure, includes any long-term goal that a user seeks to achieve. A long-term goal may include an outcome that a user doesn't seek to remedy immediately but rather seeks to remedy or achieve it at a later point in the future. For instance and without limitation, a long-term target indicator may include a desire to reverse a chronic illness or eliminate the need to manage a comprehensive diagnosis with medication. A user diagnostic factor input includes a short-term target indicator. A “short term target indicator” as used in this disclosure, includes any short-term goal that a user seeks to achieve. A short-term goal may include an outcome that a user seeks to remedy or achieve in the immediate future. A short-term target indicator may include a short term goal that a user seeks to achieve such as to reduce the number of episodes of a particular disease or a desire to rely on less medication to manage a particular comprehensive diagnosis.

With continued reference to FIG. 1, priority treatment module 140 is configured to generate a loss function utilizing a user diagnostic factor input and minimize the loss function. Mathematical expression may represent a loss function, where a “loss function” is an expression an output of which an optimization algorithm minimizes to generate an optimal result. As a non-limiting example, priority treatment module 140 may calculate variables reflecting scores relating to particular user diagnostic factor inputs, calculate an output of mathematical expression using the factor inputs, and generate an ordered treatment plan that produces an output having the lowest size, according to a given definition of “size,” of the set of outputs representing each of the plurality of ordered treatment plans; size may, for instance, included absolute value, numerical size, or the like. Selection of different loss functions may result in identification of different ordered treatment plans as generating minimal outputs; for instance, where a long-term target indicator is associated in a first loss function with a large coefficient or weight, a short-term target indicator having a small coefficient or weight for may minimize the first loss function, whereas a second loss function wherein long-term indicator has a smaller coefficient which has a larger coefficient may produce a minimal output for a different ordered treatment plan having a larger short-term indicator.

With continued reference to FIG. 1, mathematical expression and/or loss function may be generated using a machine learning to produce loss function: i.e., regression. Mathematical expression and/or loss function be user-specific, using a training set composed of past user selections; may be updated continuously. Mathematical expression and/or loss function may initially be seeded using one or more user inputs as above. User may enter a new command changing mathematical expression, and then subsequent user inputs may be used to generate a new training set to modify the new expression.

With continued reference to FIG. 1, mathematical expression and/or loss function may be generated using machine learning using a multi-user training set. Training set may be created using data of a cohort of persons having similar demographic, religious, health, and/or lifestyle characteristics to user. This may alternatively or additionally be used to seed a mathematical expression and/or loss function for a user, which may be modified by further machine learning and/or regression using subsequent user selections of alimentary provision options. Loss function analysis may measure changes in predicted values versus actual values, known as loss or error. Loss function analysis may utilize gradient descent to learn the gradient or direction that a cost analysis should take in order to reduce errors. Loss function analysis algorithms may iterate to gradually converge towards a minimum where further tweaks to the parameters produce little or zero changes in the loss or convergence by optimizing weights utilized by machine learning algorithms. Loss function analysis may examine the cost of the difference between estimated values, to calculate the difference between hypothetical and real values. Priority treatment module 140 may utilize variables to model relationships between past interactions between a user and system 100 and ordered treatment plans. In an embodiment loss function analysis may utilize variables that may impact user interactions and/or short-term target indicators and/or long-term target indicator. Loss function analysis may be user specific so as to create algorithms and outputs that are customize to variables for an individual user.

With continued reference to FIG. 1, generating an ordered treatment plan may include calculating a comprehensive diagnosis impact score where the comprehensive diagnosis impact score includes a difficulty factor multiplied by an alimentary standard factor multiplied by an implementation factor. A “comprehensive diagnosis impact score” as used in this disclosure, includes one or more attributes that may affect a diagnosis. An attribute may include any factor that may attribute to a diagnosis including how difficult it may be to seek treatment, how much money a particular treatment may cost, accessibility to receive treatment, travel time to treatment and the like. Factors may be received from user inputs, from user-client device and/or may be stored in user database. A “difficulty factor” as used in this disclosure, indicates how difficult a particular treatment facet is to implement. Difficulty factor may be rated on a numerical score ranging from 0 to 100 where 0 may indicate a particular treatment facet that is not difficult to implement and 100 may indicate a particular treatment facet that is very difficult to implement. For instance and without limitation, a user may rate a treatment facet such as adhering to a low sugar diet as 85, indicating a difficult treatment to implement while the user may rate a treatment facet such as consuming a medication once per day as 10, indicating a treatment that is not difficult to implement. An “alimentary standard factor” as used in this disclosure, indicates how expensive a particular treatment facet is to implement. Alimentary standard factors may be rated on a numerical score ranging from 0 to 100 where 0 may indicate an inexpensive treatment facet and 100 may indicate a particular treatment facet that is very expensive to implement. Alimentary standard factors may be calculated based on expert input. For instance and without limitation, a treatment facet such as a mammogram may contain an alimentary standard factor as 72 while a treatment facet such as practicing a meditation sequence may contain an alimentary standard factor as 15. An “implementation factor” as used in this disclosure, indicates how difficult a particular treatment facet is to implement into one's daily life treatment. Implementation factor may be based upon user inputs received from user database and/or user client device 160 and/or based on expert input. Implementation factor may be rated on a numerical score ranging from 0 to 100 where 0 may indicate a particular treatment facet that is not difficult to implement while a score of 100 may indicate a particular treatment facet that is difficult to implement. For instance and without limitation, a treatment facet such as implementing a fitness routine may contain an implementation factor score of 87 while a treatment facet such as taking a fish oil capsule once per day may contain an implementation factor score of 12.

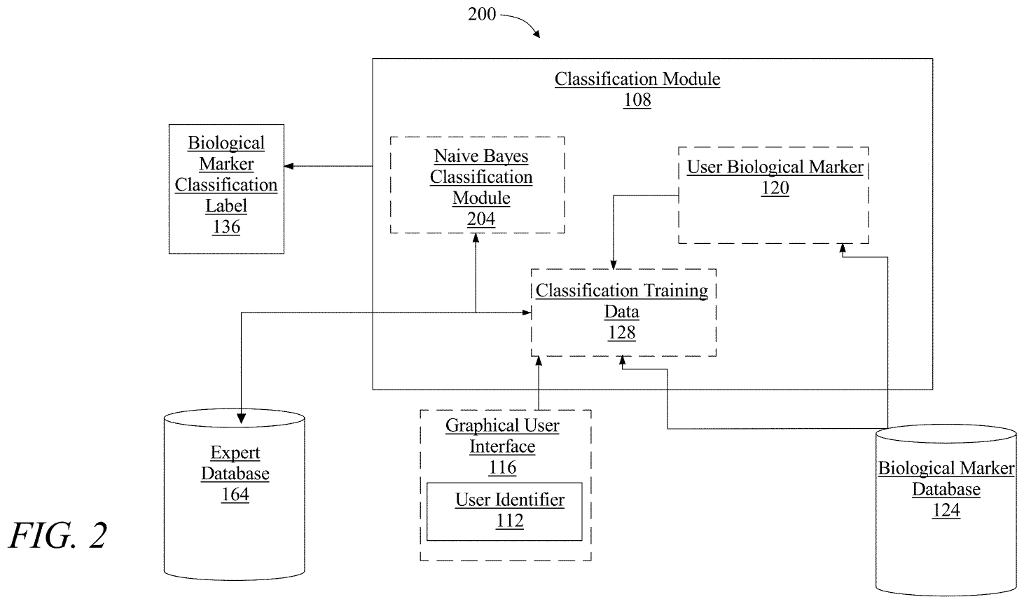

Referring now to FIG. 2, an exemplary embodiment 200 of classification module 108 is illustrated. Classification module 108 may be implemented as any hardware and/or software module. Classification module 108 receives a user identifier 142 entered by a comprehensive advisor located on graphical user interface 146. User identifier 142 may include any of the user identifier 142 as described above in reference to FIG. 1. For instance and without limitation, user identifier 142 may include a user's name and address, a particular medical record for a user, or a public private cryptographic key pair. In an embodiment, user identifier 142 may be selected from user database upon entry of information pertaining to a particular user by comprehensive advisor on graphical user interface 146. For instance and without limitation, comprehensive advisor may select a particular user from a list displayed upon graphical user interface 146 of a plurality of users, upon selecting a particular user from the list, a user identifier 142 may be retrieved from user database. In yet another non-limiting example, a user identifier 142 entered by a comprehensive advisor on graphical user interface 146 may be utilized to confirm information pertaining to a user that may be stored within user database. For example, a comprehensive advisor may enter a user's name and date of birth on graphical user interface 146, which may prompt computing device 104 and/or classification module 108 to retrieve stored information about a user within user database such as medical history, demographics, emergency contact information and the like. Stored information pertaining to a user within user database may be displayed upon graphical user interface 146 for a comprehensive advisor to confirm or edit such as when a comprehensive advisor may be having a face to face appointment with a user and seeks to update information.

With continued reference to FIG. 1, classification module 108 retrieves a user biological marker 120 from biological marker database 124. User biological marker 120 includes any of the user biological marker 120 as described above in reference to FIG. 1. For instance and without limitation, a user biological marker 120 may include a urinalysis analyzed for heavy metals including lead, iron, cadmium, mercury, aluminum, arsenic, cesium, nickel, palladium, thallium, tungsten, and uranium. In yet another non-limiting example, a user biological marker 120 may include a salivary hormone panel analyzed for one or more hormone levels including cortisol, testosterone, DHEA, estradiol, estriol, estrone, and progesterone. Biological marker 120 stored within biological marker database 124 may have been previously collected and analyzed. For instance and without limitation, biological marker database 124 may contain entries containing all biological marker 120 extracted and analyzed over the course of a user's lifestyle. Comprehensive advisor input generated through graphical user interface 146 may prompt classification module 108 to retrieve a particular biological marker 120 collected and analyzed on a particular day and/or time. In an embodiment, biological marker database 124 may contain a user identifier 142 that may be confirmed before retrieving a user biological marker 120. For instance and without limitation, a user identifier 142 such as a user's name and date of birth may be compared and matched to a user's name and date of birth associated with a particular biological marker 120.

With continued reference to FIG. 2, classification module 108 receives classification training data 128. Classification training data 128 may include any of the classification training data 128 as described above in reference to FIG. 1. Classification training data 128 includes a plurality of data entries containing biological marker 120 data containing alert and non-alert classification labels. For instance and without limitation, classification training data 128 may include a plurality of data entries containing one or more biological marker 120 data entries containing alert and non-alert classification labels. For example, a particular set of classification training data 128 may include a plurality of biological marker 120 containing varied biological marker 120 including methane breath levels, cerebrospinal neutrophil levels, salivary testosterone levels, and mercury hair levels containing alert and non-alert classification labels. In yet another non-limiting example, a particular set of classification training data 128 may include a plurality of data entries containing the same biological marker 120 containing alert and non-alert classification labels. For example, classification training data 128 may include a first salivary progesterone level containing an alert label, a second salivary progesterone level containing a non-alert classification label, a third salivary progesterone level containing an alert classification label, and a fourth salivary progesterone level containing a non-alert classification label.

With continued reference to FIG. 2, classification module 108 may receive classification training data 128 from expert database 164. Expert database 164 may be implemented as any data structure suitable for use as biological marker database 124 as described above. Classification training data 128 may be generated from expert inputs stored within expert database 164. Expert input may provide advice as to what classification training data 128 sets may be best suited to be utilized for generating algorithms for particular user biological marker 120. For instance and without limitation, a particular set of classification training data 128 may contain data entries that contain a biological marker 120 that matches a user biological marker 120. For instance and without limitation, expert input contained within expert database 164 may recommend that a particular classification training set contained within expert database 164 that includes a plurality of data entries containing a biological marker 120 such as urinary bisphenol A levels may be best suited to a user biological marker 120 that contains a urinary bisphenol A level measurement. In yet another non-limiting example, expert input contained within expert database 164 may recommend that a particular classification training set contained within expert database 164 that includes a plurality of data entries containing a biological marker 120 such as salivary progesterone may not be best suited to a user biological marker 120 that contains a plasma progesterone level.

With continued reference to FIG. 2, classification module 108 may include naïve Bayes classification module 204. Naïve Bayes classification module 204 may be implemented as any hardware and/or software module. Naïve Bayes classification module 204 generates a naïve Bayes classification algorithm utilizing classification training data 128. A naïve Bayes classification algorithm utilizes a user biological marker 120 as an input and outputs a biological marker classification label 136. Naïve Bayes classification module 204 generates a naïve Bayes classification algorithm utilizing any of the methods as described above in reference to FIG. 2. Naïve Bayes classification module 204 generates a naïve Bayes classification algorithm based on an assumption that each data entries contained within classification training set makes an independent and equal contribution to an outcome. Naïve Bayes classification module 204 generates naïve Bayes algorithm based on principles of probabilistic classifier. Naïve Bayes algorithm includes any mathematical formulas, calculations, and the like utilized to output a biological marker classification label 136. Naïve Bayes algorithm seeks to assign classification labels to problem instances which may be represented as vectors of feature values, and where classification labels may be drawn to a finite set. Naïve Bayes algorithm includes a series of calculations that assume the value of a particular data entry is independent of the value of any other feature, given a class variable. Naïve Bayes classification module 204 may be configured to calculate one or more variations of naïve Bayes algorithm including for example gaussian naïve Bayes, multinomial naïve Bayes, Bernoulli naïve Bayes, and/or semi-supervised parameter estimation. In an embodiment, naïve Bayes classification module 204 may select a particular naïve Bayes algorithm and/or series of calculations based on input from expert database 164.

With continued reference to FIG. 2, naïve Bayes classification module 108 generates an output containing biological marker classification label 136. Biological marker classification label 136 may include any of the biological marker classification label 136s as described above in reference to FIG. 1. In an embodiment, biological marker classification label 136 may include a classification label containing an alert or non-alert classification label. In an embodiment, biological marker classification label 136 may include other classification labels that may be determined based on expert input and data entries contained within classification training data 128.

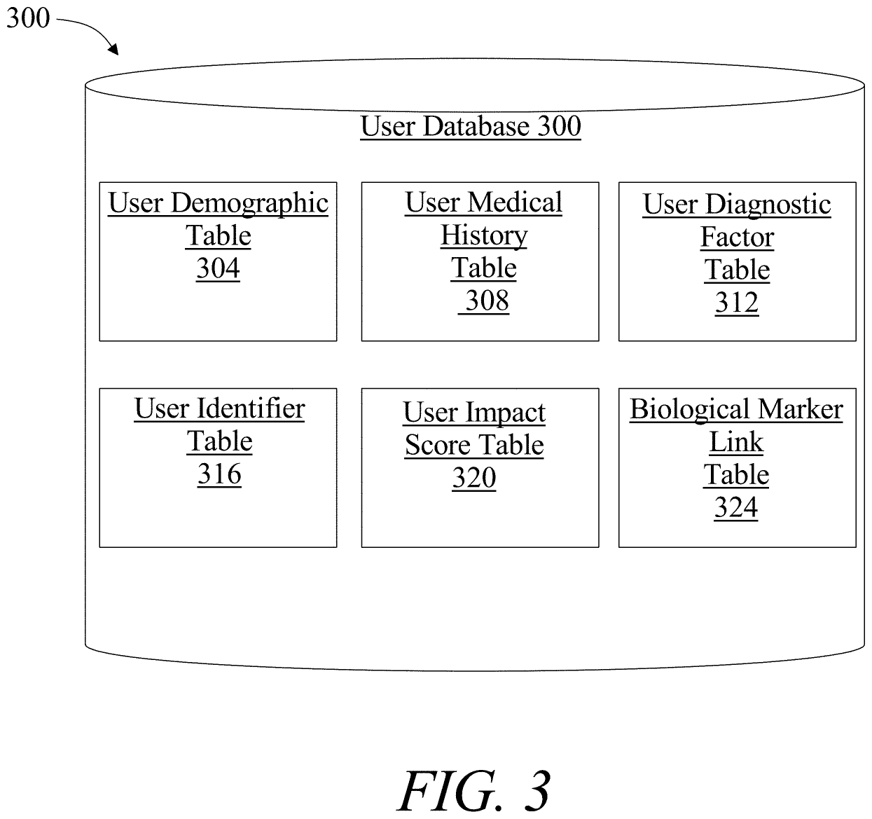

Referring now to FIG. 3, an exemplary embodiment of user database 300 is illustrated. User database 300 may be implemented as any data structure suitable for use as biological marker database 124 as described above in more detail in reference to FIG. 2. One or more tables contained within user database 300 may include user demographic table 304; user demographic table 304 may include one or more data entries relating to a user's demographics. For instance and without limitation, user demographic table 304 may include information relating to a user's full legal name, address, work address, marital status, income, occupation, and the like. One or more tables contained within user database 300 may include user medical history table 308; user medical history table 308 may include one or more data entries containing a user's medical history. For instance and without limitation, user medical history table 308 may include information describing a user's current and previous medications, allergies, surgeries, medical procedures, office visits, emergency room visits, consultations and appointments with comprehensive advisors and the like. One or more tables contained within user database 300 may include user diagnostic factor table 312; user diagnostic factor table 312 may include one or more data entries containing user diagnostic factors including long-term target indicators and short-term target indicators. For instance and without limitation, user diagnostic factor table 312 may include a current long-term target indicator entered by a user and a current short-term target indicator relating to a particular comprehensive diagnosis. One or more tables contained within user database 300 may include user identifier 142 table 316; user identifier 142 table 316 may include one or more data entries containing one or more user identifier 142. For instance and without limitation, user identifier 142 table 316 may include user identifier 142 that include a user's name, address, date of birth, public-private cryptographic key pairs, biometric authentications and the like. One or more tables contained within user database 300 may include user impact score table 320; user impact score 320 may include one or more data entries containing a user impact score. For instance and without limitation, user impact score table 320 may include a user impact score generated by a user in regards to a particular treatment facet. One or more tables contained within user database 300 may include biological marker 120 link table 324; biological marker 120 link table 324 may include one or more links of user data contained within user database 300 to information contained within biological marker database 124.

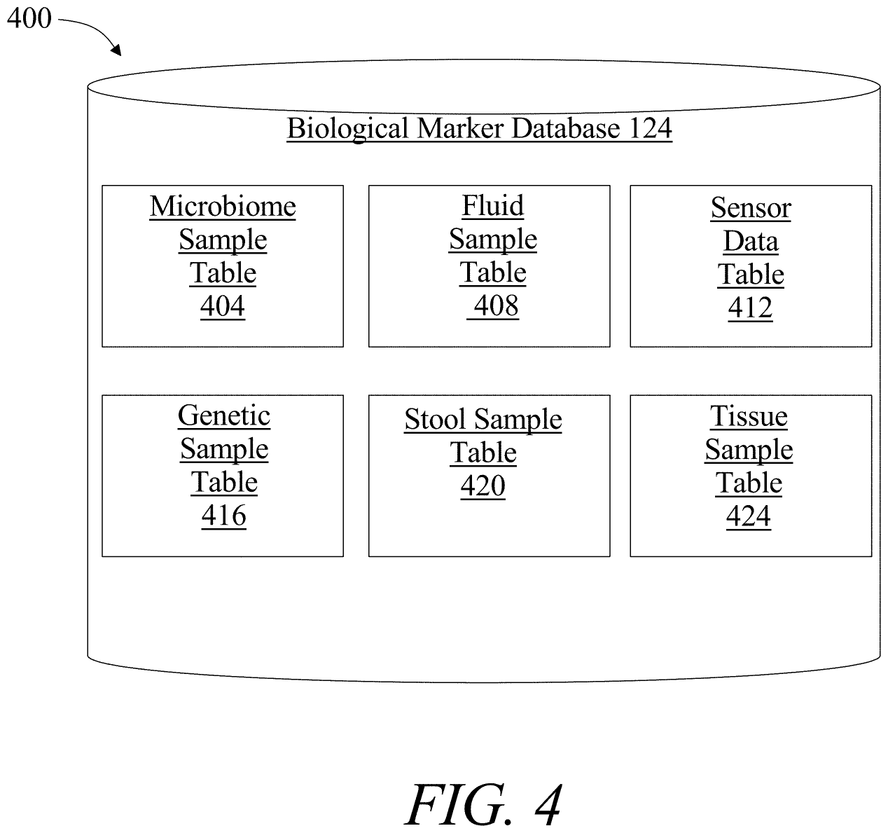

Referring now to FIG. 4, an exemplary embodiment 400 of biological marker database 124 is illustrated. Biological marker database 124 may include any data structure as described above in reference to FIG. 1. Biological marker database 124 may store one or more biological marker 120. One or more tables contained within biological marker database 124 may include microbiome sample table 404; microbiome sample table 404 may store one or more biological marker 120 relating to the microbiome. For instance and without limitation, microbiome sample table 404 may include results reflecting levels of a particular bacterial strain such as quantities of Bifidobacterium found in a user's gastrointestinal tract. One or more tables contained within biological marker database 124 may include fluid sample table 408; fluid sample table 408 may store one or more biological marker 120 obtained from a fluid sample. For instance and without limitation, fluid sample table 408 may include one or more entries containing results from fluids such as urine, saliva, sweat, tears, blood, mucus, cerebrospinal fluid, and the like analyzed for one or more biological marker 120. One or more tables contained within biological marker database 124 may include sensor data table 412; sensor data table 412 may include one or more biological marker 120 obtained from one or more sensors. For instance and without limitation, sensor data table 412 may include sleeping patterns of a user recorded by a sensor. One or more tables contained within biological marker database 124 may include genetic sample table 416; genetic sample table 416 may include one or more biological marker 120 containing one or more genetic sequences. For instance and without limitation, genetic sample table 416 may include a user's genetic sequence for a particular gene such as a sequence illustrating a positive breast cancer one (BRACA 1) gene. One or more tables contained within biological marker database 124 may include stool sample table 420; stool sample table 420 may include one or more biological marker 120 obtained from a stool sample. For instance and without limitation, stool sample table 420 may include a user's stool sample analyzed for the presence and/or absence of one or more parasites. One or more tables contained within biological marker database 124 may include tissue sample table 424; tissue sample table 424 may include one or more biological marker 120 obtained from one or more tissue samples. For instance and without limitation, tissue sample table 424 may include a breast tissue sample analyzed for the absence and/or presence of estrogen markers. Other tables not illustrated may include but are not limited to epigenetic, gut wall, nutrients, metabolism, and user database link table.

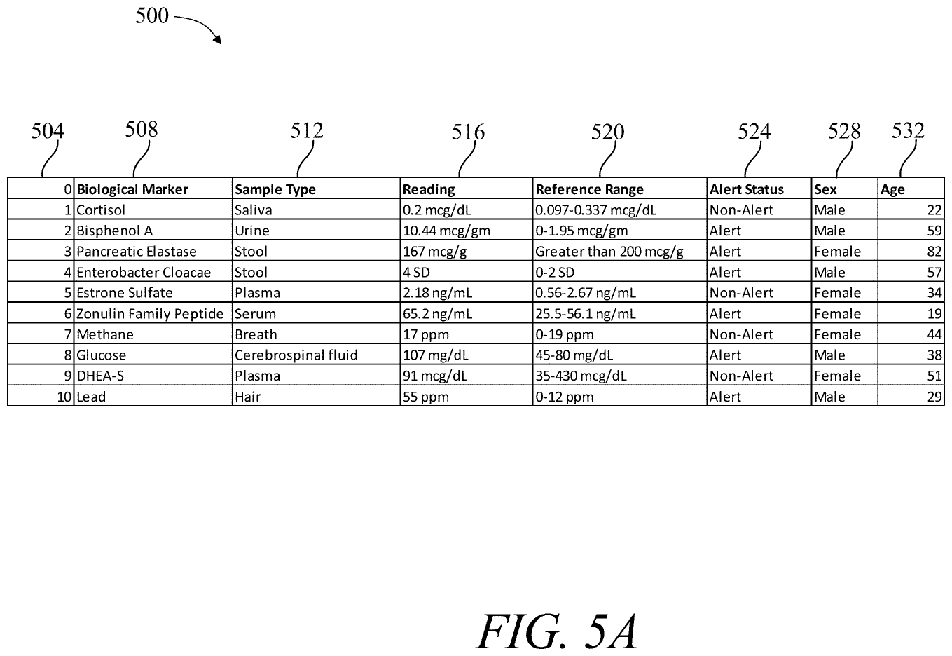

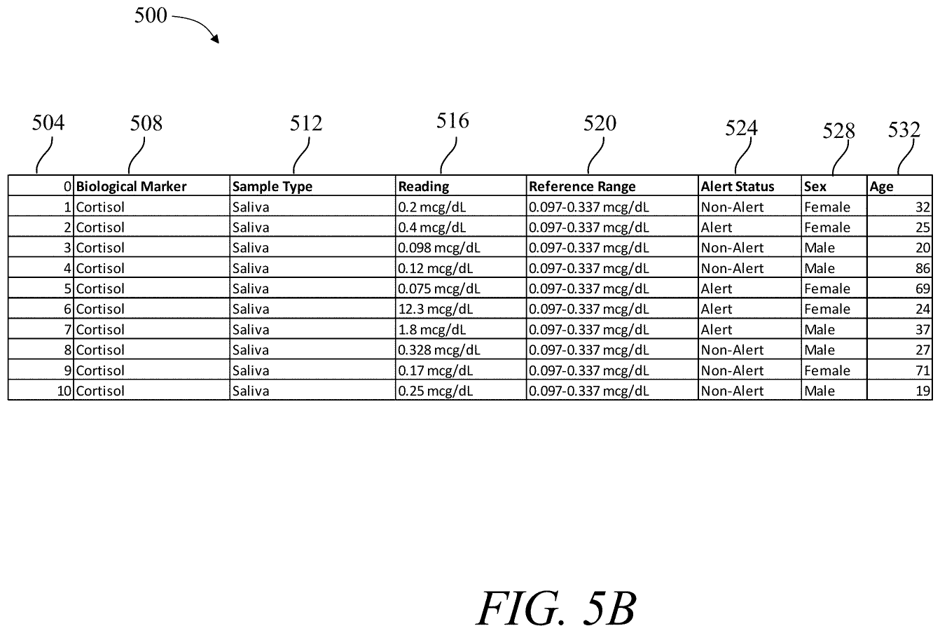

Referring now to FIGS. 5A-5B, an exemplary embodiment 500 of classification training data 128 is illustrated. Referring now to FIG. 5A, an embodiment of classification training data 128 containing a plurality of biological makers is illustrated. In an embodiment, classification training data 128 may include a plurality of data entries 504. Each data entry 504 may include a particular identified biological marker 120 508. In FIG. 5A, data entries 504 each contain varying biological marker 120. For instance and without limitation, a biological marker 120 may include cortisol, bisphenol A, pancreatic elastase, Enterobacter cloacae, estrone sulfate, zonulin family peptide, methane, glucose, DHEA-S, lead, and the like. Each data entry 504 may indicate a particular sample type 512 of each biological marker 120 508 identified. For instance and without limitation, a biological marker 120 508 such as cortisol may be obtained and analyzed from saliva, blood, urine, and tissue samples. Sample type 512 indicates which particular sample from a human body was extracted. Each data entry 504 may include a particular reading 516 or measurement obtained from a particular sample. Each data entry 504 may include a reference range 520 for each particular biological marker 120 508 which may be adjusted based on what particular sample type 512 was extracted. For example, a reference range 520 for plasma progesterone levels may be different than a reference range 520 for salivary progesterone levels. Each data entry 504 contains an alert status 524 which includes a classification label containing an alert or non-alert classification label. Each data entry 504 contains a gender identifier 528 which indicates what sex the data entry 504 was received from. Each data entry 504 contains an age identifier 532 which indicates what age the data entry 504 was received from.

Referring now to FIG. 5B, an embodiment of classification training data 128 containing one type of biological marker 120 504 is illustrated. In an embodiment, classification training data 128 may include a plurality of data entries 504. Each data entry 504 may include the same identified biological marker 120 508. In FIG. 5B, data entries 504 each contain the same biological marker 120 508, cortisol. Each data entry 504 may indicate a particular sample type 512 of each biological marker 120 508 identified. In an embodiment, sample type 512 may be the same, such as in FIG. 5B where the sample type is exclusively saliva. Sample type 512 indicates which particular sample from a human body was extracted. Each data entry 504 may include a particular reading 516 or measurement obtained from a particular sample. Each data entry 504 may include a reference range 520 for each particular biological marker 120 508 which may be adjusted based on what particular sample type 512 was extracted. Each data entry 504 contains an alert status 524 which includes a classification label containing an alert or non-alert classification label. Each data entry 504 contains a gender identifier 528 which indicates what sex the data entry 504 was received from. Each data entry 504 contains an age identifier 532 which indicates what age the data entry 504 was received from. In an embodiment, various combinations of biological marker 120, sample types, sex, age, and other variables containing within classification training set are possible.

With continued reference to FIGS. 5A-5B, classification training data 128 may be stored in any suitable data and/or data type. For instance and without limitation, clustering dataset may include textual data such as numerical, character, and/or string data. Textual data may include a standardized name and/or code for a disease, disorder, or the like; codes may include diagnostic codes and/or diagnosis codes, which may include without limitation codes used in diagnosis classification systems such as The International Statistical Classification of Diseases and Related Health Problems (ICD). In general, there is no limitation on forms textual data or non-textual data used as dataset may take; persons skilled in the art, upon reviewing the entirety of this disclosure, will be aware of various forms which may be suitable for use as dataset consistently with this disclosure.

With continued reference to FIG. 5, classification training data 128 may be stored as image data, such as for example an image of a particular food substance such as a photograph of a pear or an image of a steak. Image data may be stored in various forms including for example, joint photographic experts group (JPEG), exchangeable image file format (Exif), tagged image file format (TIFF), graphics interchange format (GIF), portable network graphics (PNG), netpbm format, portable bitmap (PBM), portable any map (PNM), high efficiency image file format (HEIF), still picture interchange file format (SPIFF), better portable graphics (BPG), drawn filed, enhanced compression wavelet (ECW), flexible image transport system (FITS), free lossless image format (FLIF), graphics environment manage (GEM), portable arbitrary map (PAM), personal computer exchange (PCX), progressive graphics file (PGF), gerber formats, 2 dimensional vector formats, 3 dimensional vector formats, compound formats including both pixel and vector data such as encapsulated postscript (EPS), portable document format (PDF), and stereo formats.

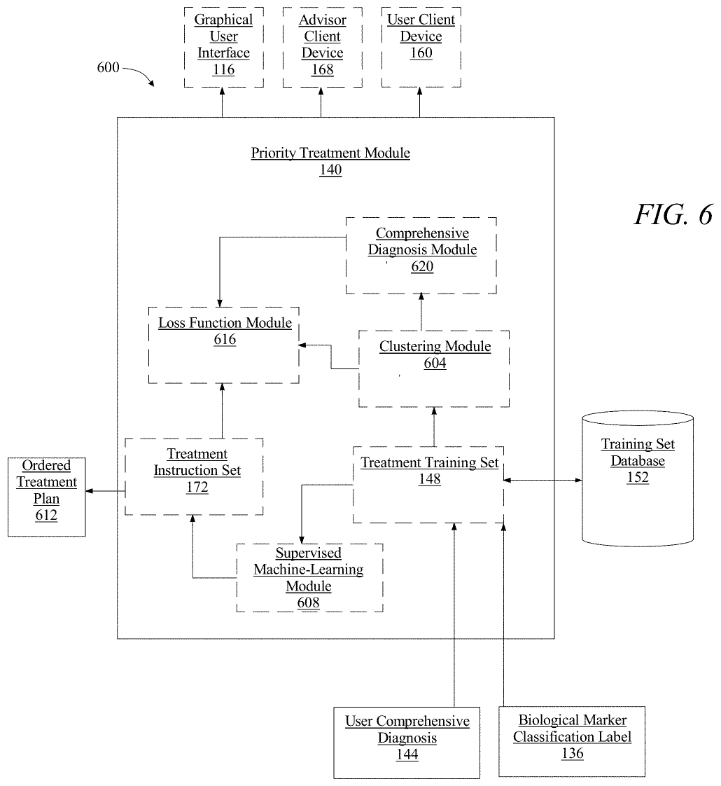

Referring now to FIG. 6, an exemplary embodiment 600 of priority treatment module 140 is illustrated. Priority treatment module 140 may include any hardware and/or software module. Priority treatment module 140 is configured to receive a biological marker classification label 136 from classification module 108. Priority treatment module 140 receives a biological marker classification label 136 from classification module 108 utilizing any network topography as described herein. Priority treatment module 140 receives a plurality of user comprehensive diagnoses 144. User comprehensive diagnoses 144 include any of the user comprehensive diagnoses 144 as described above in reference to FIG. 1. Plurality of user comprehensive diagnoses 144 relate to the same user. For instance and without limitation, a plurality of user comprehensive diagnoses 144 may include rheumatoid arthritis, back strain, hearing loss, and Alzheimer's disease. In yet another non-limiting example, a plurality of user comprehensive diagnoses 144 may include estrogen dominance, generalized anxiety disorder, and leaky gut. User comprehensive diagnoses 144 may be entered by comprehensive advisor through a graphical user interface 146. In yet another non-limiting example, comprehensive advisor may select one or more comprehensive diagnoses that may be stored within user database.

With continued reference to FIG. 6, priority treatment module 140 selects a treatment training set 148. Treatment training set 148 may be stored within training set database 152. Training set database 152 may be implemented as any data structure suitable for use as biological marker database 124 as described above. Treatment training set 148 contains a plurality of data entries containing comprehensive diagnoses correlated to one or more prioritized treatment facets. Prioritized treatment facets include any of the prioritized treatment facets as described above in reference to FIG. 1. Prioritized treatment facet includes an identification of what aspect of a comprehensive diagnosis needs to be addressed and treated in a sequential manner. For instance and without limitation, prioritized treatment facet for a comprehensive diagnosis such as small intestinal bacterial overgrowth (SIBO) may include a first facet that includes eradicating overgrowth of bacteria in small intestine, a second facet that includes addressing nutritional deficiencies, a third facet that includes initiating a low-FODMAP diet, and a fourth facet that includes repopulating beneficial bacteria.

With continued reference to FIG. 6, priority treatment module 140 selects a treatment training set 148 as a function of biological marker classification label 136. Priority treatment module 140 may select a treatment training set 148 that contains a classifier label that matches a biological marker classification label 136. Treatment training set 148 contained within training set database 152 may be organized according to various classifier labels. Treatment training set 148 may be organized within training set database 152 by classifier labels such as by alert or non-alert classifier or by type of comprehensive diagnosis training data contained within a particular treatment training set 148. Matching may include determining that a classifier label contained within training set database 152 is the same as a biological marker classification label 136. For example, a biological marker classification label 136 that contains an alert classifier label may be matched to a treatment training set 148 that contains an alert classifier label. In yet another non-limiting example, a biological marker classification label 136 that contains a classifier label by comprehensive diagnosis that contains a comprehensive diagnosis classifier label of ulcerative colitis may be matched to a treatment training set 148 that contains a classifier label of ulcerative colitis. In an embodiment, treatment training set 148 may contain classifier labels generated by expert input such as from information stored and contained within expert database 164.

With continued reference to FIG. 6, priority treatment module 140 may include clustering module 604, which may be implemented as any hardware and/or software module. Clustering module 604 may be configured to receive diagnostic training data where diagnostic training data contains a plurality of data entries including comprehensive diagnoses including urgent and non-urgent classification labels. For instance and without limitation, diagnostic training data may include a plurality of data entries containing gout containing an urgent classification label, tension headache containing a non-urgent classification label, arthritis containing a non-urgent classification label, and myocardial infarction containing an urgent classification label. Clustering module 604 may receiving diagnostic training data from training set database 152. Clustering module 604 may be configured to generate a classification algorithm utilizing diagnostic training data. Classification algorithm may include any classification algorithm including for example but not limited to logistic regression, naïve Bayes classifier, k-nearest neighbor, support vector machines, decision trees, boosted trees, random forest, and/or neural networks. Classification algorithm utilizes a plurality of user comprehensive diagnoses 144 as input and outputs a comprehensive diagnosis classification label for each of the plurality of user comprehensive diagnoses 144. Comprehensive diagnosis classification label may include any identifier indicating if a particular comprehensive diagnosis belongs to a particular class or not. Clustering module 604 may utilize comprehensive diagnosis classification label to select a treatment training set 148 for each of the plurality of user comprehensive diagnoses 144. In an embodiment, a particular cluster containing a comprehensive diagnosis classification label may be utilized as treatment training set 148. In an embodiment, comprehensive diagnosis classification label may be matched to a treatment training set 148 containing the same classification label. Comprehensive diagnosis classification labels may be utilized to generate a treatment instruction set 172 as described in more detail below.

With continued reference to FIG. 6, priority treatment module 140 may include supervised machine-learning module 608, which may be implemented as any hardware and/or software module. Supervised machine-learning module 608 generates using a supervised machine-learning model a treatment 156 that outputs an ordered priority treatment list for each of the plurality of comprehensive diagnoses utilizing the selected treatment training set. Supervised machine-learning model includes any of the supervised machine-learning models as described above in reference to FIG. 1. Treatment 156 may include any machine learning process and may include linear or polynomial regression algorithms. Treatment 156 may include equations. Treatment 156 may include a set of instructions utilized to generate outputs based on inputs derived using a machine-learning algorithm and the like. Ordered priority treatment list includes any of the ordered priority treatment list as described above. Ordered priority treatment list may include one or more treatment facets arranged in a sequential manner. For instance and without limitation, an ordered priority treatment list for a comprehensive diagnosis such as estrogen dominance may include a first treatment facet that includes initiating a daily sauna practice, a second treatment facet that includes initiating an exercise regimen, a third treatment facet that includes using clean beauty products, a fourth treatment facet that includes initiating an estrogen dominance diet, and a fifth treatment facet that includes initiating an estrogen blocking supplement regimen.

With continued reference to FIG. 6, priority treatment module 140 evaluates ordered priority treatment list for each of the plurality of comprehensive diagnoses generated by supervised machine-learning module. Evaluating ordered priority treatment list may include determining by priority treatment module 140 how a particular priority treatment considers a user's symptoms. Priority treatment module 140 may retrieve an element of user symptom data from user database. An element of user symptom data may include any of the elements of user symptom data as described above in reference to FIG. 1. Priority treatment module 140 may correlate an element of user symptom data to a comprehensive diagnosis. Correlation may be performed utilizing any of the methods as described above in reference to FIG. 1. Priority treatment module 140 may utilize the correlated element of user symptom data to a comprehensive diagnosis to generate an ordered treatment plan 612.

With continued reference to FIG. 6, priority treatment module 140 may include loss function module 616, which may be implemented as any hardware and/or software module. Loss function module 616 may aid in generating a treatment instruction set 172 by retrieving a user diagnostic factor input from user database. User diagnostic factor input includes any user diagnostic factor input as described above in reference to FIG. 1. User diagnostic factor input includes a long-term target indicator and a short-term target indicator. For instance and without limitation, user diagnostic factor input includes a long-term target indicator such as a desire to reverse a user's comprehensive diagnosis of Type 2 Diabetes Mellitus and a short-term target indicator such as a desire to eliminate the number of hypoglycemic episodes that a user experiences each week. Loss function module generates a loss function and minimizes the loss function utilizing the user diagnostic factor input containing a long term target indicator and a short term target indicator. Loss function may be generated utilizing any of the methods as described above in reference to FIG. 1. Loss function module generates an ordered treatment plan as a function of minimizing the loss function. For instance and without limitation, a particular short-term target indicator may be utilized to generate an ordered treatment plan 612 that contains a particular treatment facet in a particular order or in front of or behind a particular second treatment facet.

With continued reference to FIG. 6, priority treatment module 140 is configured to generate a treatment instruction set. Treatment instruction set 172 includes any of the treatment instruction sets as described above in reference to FIG. 1. Treatment instruction set 172 includes an ordered treatment plan 612. An “ordered treatment plan” as used in this disclosure, includes a stepwise treatment plan for two or more comprehensive diagnoses that contains a chronological sequence of prioritized treatment facets for the two or more comprehensive diagnoses. For instance and without limitation, a first comprehensive diagnosis such as gallstones may contain prioritized treatment facets that includes a first facet recommending an anti-inflammatory diet, a second facet recommending a gallbladder flush, and a third facet recommending a fish oil supplement. A second comprehensive diagnosis such as heart disease may include a first facet that recommends taking a red rice yeast extract supplement and a second facet that recommends an anti-inflammatory diet. In such an instance, ordered treatment plan may include a stepwise treatment plan for the gallstones and the heart disease and may contain a first facet recommending an anti-inflammatory diet, a second facet recommending a gallbladder flush, a third facet recommending taking a red rice yeast extract supplement, and a fourth facet recommending taking a fish oil supplement. Facets may be ordered within ordered treatment plan based on expert inputs and machine-learning algorithms including any of the machine-learning algorithms as descried herein. Generating an ordered treatment plan may include selecting a first treatment facet as a function of a first priority treatment, selecting a second treatment facet as a function of the first treatment facet and selecting a third treatment facet as function of the second treatment facet. Generating an ordered treatment plan may include selecting a first treatment facet as a function of a first priority treatment, selecting a second treatment facet as a function of the first priority treatment, and selecting a third treatment facet as a function of the second priority treatment. Generating an ordered treatment plan may include receiving a comprehensive input descriptor generated by a comprehensive advisor that contains an advisor interaction summary. Advisor interaction summary may include any of the advisor interaction summaries as described above in more detail in reference to FIG. 1. For instance and without limitation, advisor interaction summary may include a description of a phone call between a comprehensive advisor and a user when user complained of gastrointestinal symptoms including gas, explosive diarrhea, and fatigue. In yet another non-limiting example, comprehensive advisor input may include an advisor interaction summary that includes a description of a video chat between a comprehensive advisor and a user when user described a lack of motivation and extreme sadness. Ordered treatment plan 612 may be generated based on advisor interaction summaries such as by selecting a particular treatment facet to be at a specific point in a sequential sequence of treatment facets.

With continued reference to FIG. 6, priority treatment module 140 may include comprehensive diagnosis module 620, which may be implemented as any hardware and/or software module. Comprehensive diagnosis module 620 may calculate comprehensive diagnosis impact score which may be utilized to generate treatment instruction set. Comprehensive diagnosis impact score includes a difficulty factor multiplied by an alimentary standard factor multiplied by an implementation factor. Comprehensive diagnosis impact score may include any of the comprehensive diagnosis impact scores as described above in more detail in reference to FIG. 1. Difficult factors, alimentary standard factors, and implementation factors may be obtained from user inputs received from user database, user client device 160, as well as from advisory inputs and advisor client device 168.

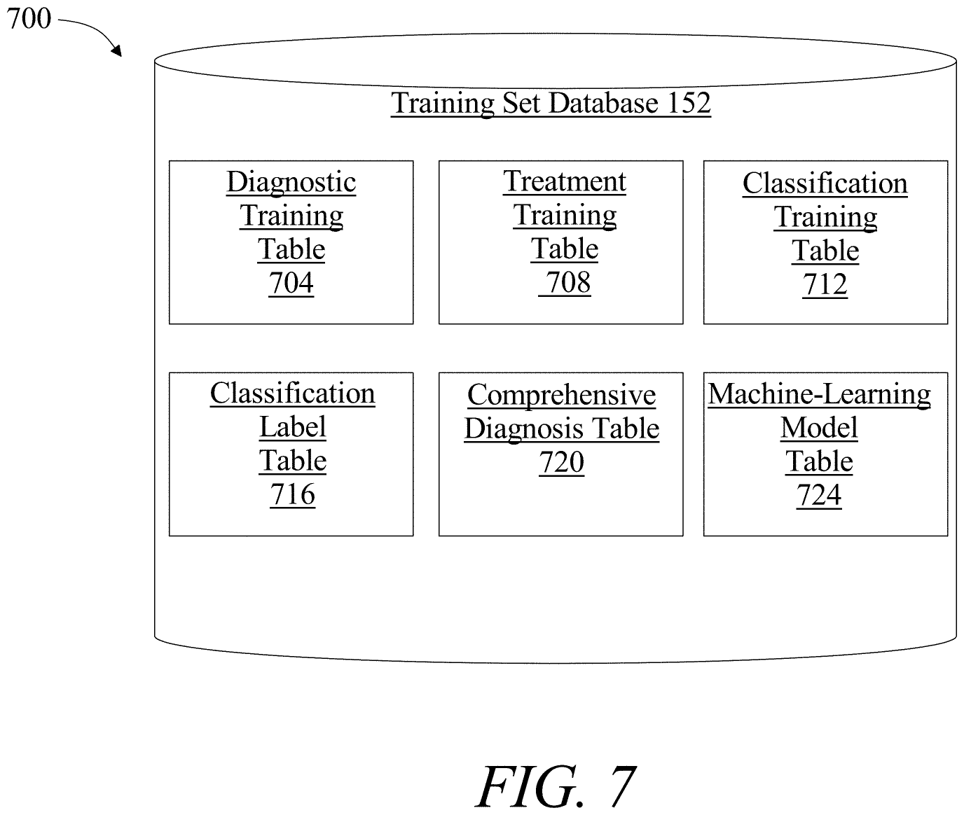

Referring now to FIG. 7, an exemplary embodiment of training set database 152 is illustrated. Training set database 152 may be implemented as any data structure suitable for use as biological marker database 124 as described in more detail in reference to FIG. 1. One or more tables contained within training set database 152 may include diagnostic training table 704; diagnostic training table 704 may include one or more data entries containing one or more diagnostic training sets. One or more tables contained within training set database 152 may include treatment training table 708; treatment training table 708 may include one or more data entries containing one or more treatment training set 148. One or more tables contained within training set database 152 may include classification training table 712; classification training table 712 may include one or more data entries containing one or more classification training sets. One or more tables contained within training set database 152 may include classification label table 716; classification label table 716 may contain one or more training sets organized by classification labels. One or more tables contained within training set database 152 may include comprehensive diagnosis table 720; comprehensive diagnosis table 720 may include one or more training sets organized by comprehensive diagnosis. One or more tables contained within training set database 152 may include machine-learning model table 724; machine-learning model table 724 may include one or more machine-learning models that may have been previously calculated and ready to be utilized.

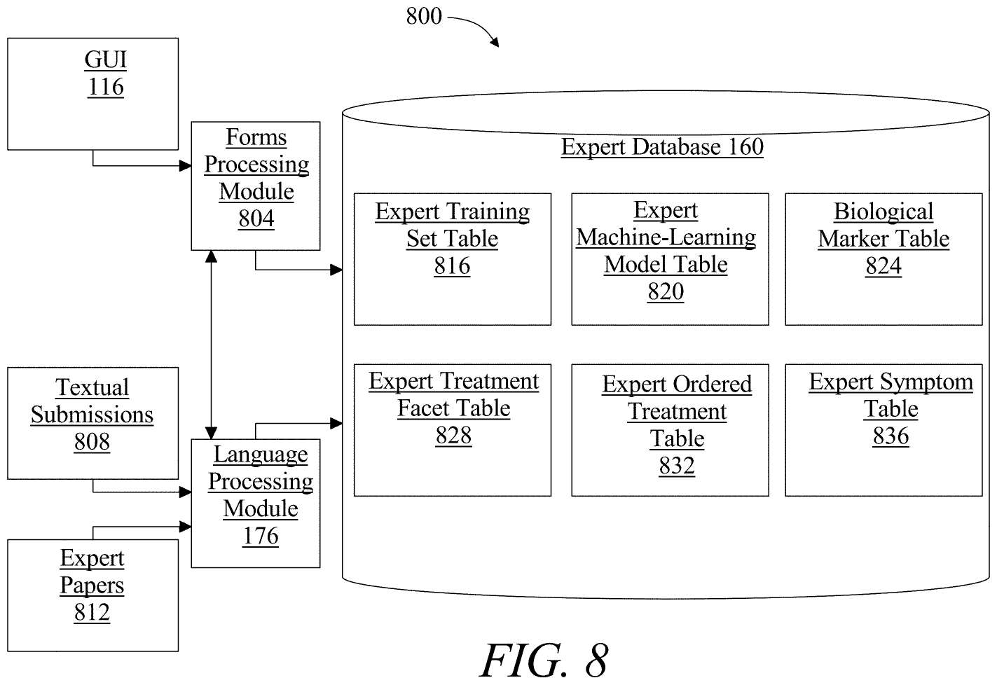

Referring now to FIG. 8, an exemplary embodiment of expert database 164 is illustrated. Expert database 164 may be implemented as any data structure suitable for use as biological marker database 124 as described above in more detail in reference to FIG. 1. One or more database tables may be linked to one another by, for instance, common column values. For instance, a common column between two tables of expert database 164 may include an identifier of an expert submission, such as a form entry, textual submission, expert paper, or the like, for instance as defined below; as a result, a query may be able to retrieve all rows from any table pertaining to a given submission or set thereof. Other columns may include any other category usable for organization or subdivision of expert data, including types of expert data, names and/or identifiers of experts submitting the data, times of submission, or the like; persons skilled in the art, upon reviewing the entirety of this disclosure, will be aware of various ways in which expert data may be included in one or more tables.

With continued reference to FIG. 8, expert database 164 includes a forms processing module 804 that may sort data entered in a submission via graphical user interface 146 by, for instance, sorting data from entries in the graphical user interface 146 to related categories of data; for instance, data entered in an entry relating in the graphical user interface 146 to a biological marker 120 may be sorted into variables and/or data structures for storage of biological marker 120, while data entered in an entry relating to a category of training data and/or an element thereof may be sorted into variables and/or data structures for the storage of, respectively, categories of training data. Where data is chosen by an expert from pre-selected entries such as drop-down lists, data may be stored directly; where data is entered in textual form, language processing module 176 may be used to map data to an appropriate existing label, for instance using a vector similarity test or other synonym-sensitive language processing test to map physiological data to an existing label. Alternatively or additionally, when a language processing algorithm, such as vector similarity comparison, indicates that an entry is not a synonym of an existing label, language processing module 176 may indicate that entry should be treated as relating to a new label; this may be determined by, e.g., comparison to a threshold number of cosine similarity and/or other geometric measures of vector similarity of the entered text to a nearest existent label, and determination that a degree of similarity falls below the threshold number and/or a degree of dissimilarity falls above the threshold number. Data from expert textual submissions 808, such as accomplished by filling out a paper or PDF form and/or submitting narrative information, may likewise be processed using language processing module 176. Data may be extracted from expert papers 812, which may include without limitation publications in medical and/or scientific journals, by language processing module 176 via any suitable process as described herein. Persons skilled in the art, upon reviewing the entirety of this disclosure, will be aware of various additional methods whereby novel terms may be separated from already-classified terms and/or synonyms therefore, as consistent with this disclosure.

With continued reference to FIG. 8, one or more tables contained within expert database 164 may include expert training set table 816; expert training set table 816 may include one or more expert inputs regarding training sets. One or more tables contained within expert database 164 may include expert machine-learning model table 820; expert machine-learning model table 820 may include one or more expert inputs regarding machine-learning models. One or more tables contained within expert database 164 may include biological marker 120 table 824; biological marker 120 table 824 may include one or more expert inputs regarding biological marker 120. One or more tables contained within expert database 164 may include expert treatment facet table 828; expert treatment facet table 828 may include one or more expert inputs regarding treatment facets. One or more tables contained within expert database 164 may include expert ordered treatment table 832; expert ordered treatment table 832 may include one or more expert inputs regarding ordered treatment. One or more tables contained within expert database 164 may include expert symptom table 836; expert symptom table 836 may include one or more expert inputs regarding symptoms.

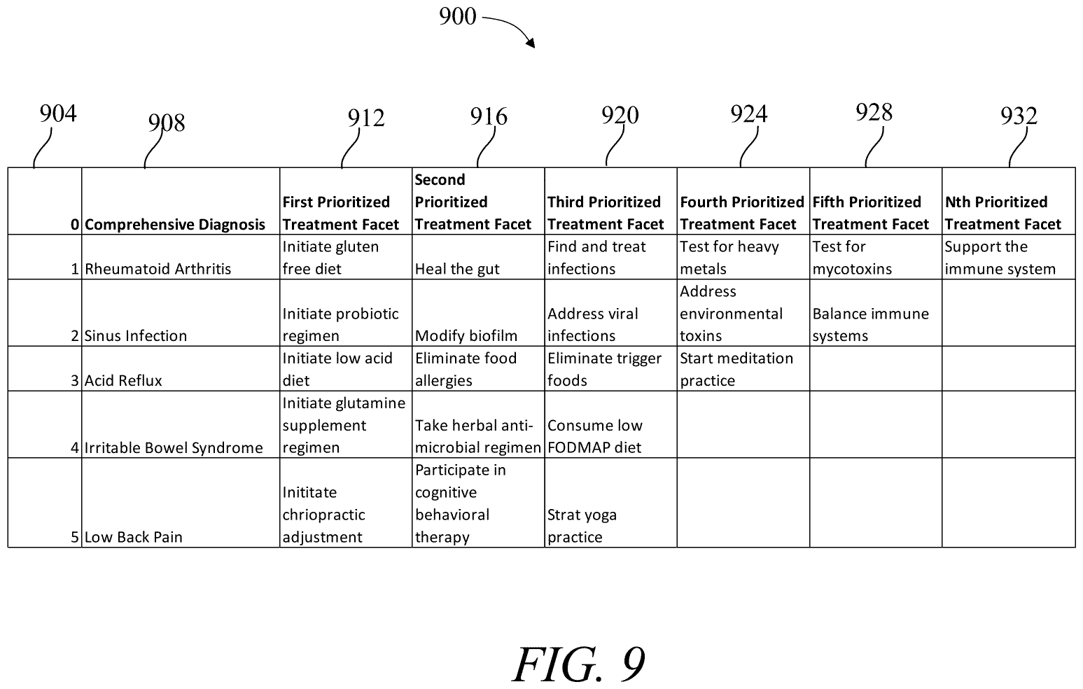

Referring now to FIG. 9, an exemplary embodiment 900 of treatment training set is illustrated. Treatment training set includes a plurality of data entries 904. Each data entry 904 contains a comprehensive diagnosis 908 correlated to one or more prioritized treatment facets. In an embodiment, a comprehensive diagnosis 908 may be correlated to a first prioritized treatment facet 912. In an embodiment, a comprehensive diagnosis 908 may be correlated to a second prioritized treatment facet 916. In an embodiment, a comprehensive diagnosis 908 may be correlated to a third prioritized treatment facet 920. In an embodiment, a comprehensive diagnosis 908 may be correlated to a fourth prioritized treatment facet 924. In an embodiment, a comprehensive diagnosis 908 may be correlated to a fifth prioritized treatment facet 928. In an embodiment, a comprehensive diagnosis 908 may be correlated to a Nth prioritized treatment facet 932. For instance and without limitation, treatment training set 148 may contain a data entry 904 containing a comprehensive diagnosis 908 such as rheumatoid arthritis that may be correlated to a first prioritized treatment facet 912 initiate gluten free diet, a second prioritized treatment facet 916 heal the gut, a third prioritized treatment facet 920 find and treat infections, a fourth prioritized treatment facet 924 test for heavy metals, a fifth prioritized treatment facet 928 test for mycotoxins, and an Nth prioritized treatment facet support the immune system.

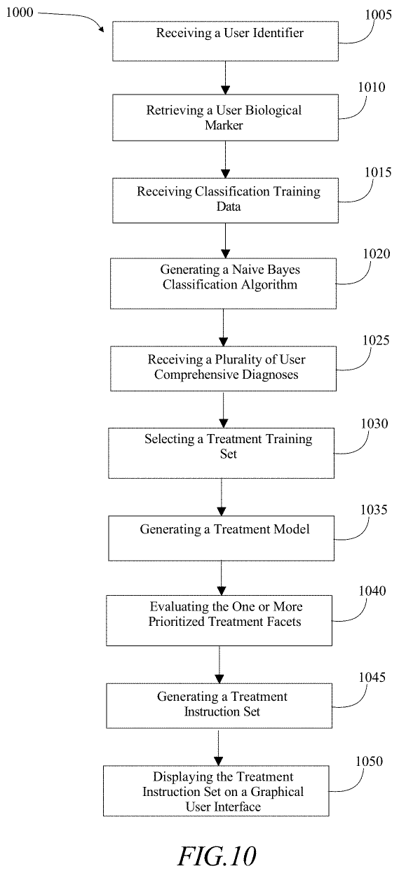

Referring now to FIG. 10, an exemplary embodiment of a method 1000 of prioritizing comprehensive diagnoses is illustrated. At step 1005 a processor receives a user identifier 142 entered by a comprehensive advisor located on a graphical user interface 146 operating on the processor. A user identifier 142 includes any of the user identifier 142 as described above in reference to FIG. 1. For instance and without limitation, a user identifier 142 may include a piece of information identifying a particular user such as a user's name or date of birth. Comprehensive advisor includes any of the comprehensive advisors as described above in reference to FIG. 1. For instance and without limitation, comprehensive advisor may include a functional medicine doctor, nurse practitioner, physician assistant and the like. In an embodiment, comprehensive advisor may enter a user identifier 142 on graphical user interface 146 and more information pertaining to a user may be retrieved from a user database. User database may include background information about a user such as a user's demographics, previous medical history, and the like.

With continued reference to FIG. 10, at step 1010 a processor retrieves a user biological marker 120 from a biological marker database 124 as a function of a user identifier 142. User biological marker 120 includes any of the user biological marker 120 as described above in reference to FIG. 1. Biological marker database 124 may contain biological marker 120 pertaining to a user as described above in reference to FIGS. 1-9. Processor may retrieve a biological marker 120 from biological marker database 124 by matching the user identifier 142 receive from comprehensive advisor to a user identifier 142 stored within biological marker database 124. For instance and without limitation, processor may match a user's name and address entered by comprehensive advisor on graphical user interface 146 to a user's name and address stored within biological marker database 124 to ensure that contain the same information. Processor may retrieve a particular user biological marker 120 based on input from comprehensive advisor located on graphical user interface 146. For example, user identifier 142 entered by comprehensive advisor may specify a particular biological extraction to retrieve or a particular time period to retrieve a stored biological marker 120 from.

With continued reference to FIG. 10, at step 1015 a processor receives classification training data 128 wherein classification training data 128 contains a plurality of data entries including biological marker 120 data containing alert and non-alert classification labels. Classification training data 128 may include any of the classification training data 128 as described above in reference to FIGS. 1-9. Classification training data 128 may be generated and received based on expert input as described above in more detail in reference to FIGS. 1-9. Classification training data 128 may be received from expert database 164 and/or training set database 152.

With continued reference to FIG. 10, at step 1020 a processor generates a naïve Bayes classification algorithm utilizing classification training data 128 wherein the naïve Bayes classification algorithm utilizes the user biological marker 120 as an input and outputs a biological marker classification label 136. Generating a naïve Bayes classification algorithm may be performed utilizing any of the methods as described above in reference to FIGS. 1-9. Biological marker classification label 136 includes any of the biological marker classification label 136s as described above in reference to FIGS. 1-9. In an embodiment, biological marker classification label 136 may contain a classifier indicator of an alert or non-alert. For instance and without limitation, naïve Bayes classification algorithm may generate a biological marker classification label 136 for a biological maker such as a serum fasting blood glucose level that contains an alert biological marker classification label 136 while a urinary glucose level that contains a non-alert biological marker classification label 136.

With continued reference to FIG. 10, at step 1025 a processor receives a plurality of user comprehensive diagnoses 144 entered by a comprehensive advisor on a graphical user interface 146 operating on the processor. A plurality of comprehensive diagnoses may include any of the comprehensive diagnoses as described above in reference to FIGS. 1-10. In an embodiment, comprehensive advisor may select one or more comprehensive diagnoses displayed on a graphical user interface 146 pertaining to a user. In yet another non-limiting example, one or more comprehensive diagnoses pertaining to a user may be stored within user database. User database is described above in more detail in reference to FIGS. 1-10. In an embodiment, a processor may display one or more comprehensive diagnoses retrieved from user database, whereby comprehensive advisor may select one or more comprehensive diagnoses on graphical user interface 146 by highlighting one or more that are displayed or touching the screen surface on the graphical user interface 146. In yet another non-limiting example, comprehensive advisor may enter in free form textual input boxes one or more comprehensive diagnoses at graphical user interface 146. In yet another non-limiting example, comprehensive advisor may select one or more comprehensive diagnoses from a drop down list displayed on graphical user interface 146.

With continued reference to FIG. 10, at step 1030 a processor selects a treatment training set 148 as a function of a biological marker classification label 136 wherein the treatment training set 148 includes a plurality of data entries containing comprehensive diagnoses correlated to one or more prioritized treatment facets. Treatment training set 148 may include any of the treatment training set 148 as described above in reference to FIGS. 1-9. A processor may select a treatment training set 148 that contains a classifier label that matches a biological marker classification label 136. For instance and without limitation, a processor may select a treatment training set 148 from training set database 152 that contains a classifier label such as “alert” that matches a biological marker classification label 136 such as “alert.” Treatment training set 148 may be organized within training set database 152 according to classification labels as described above in more detail in reference to FIGS. 1-9.

With continued reference to FIG. 10, selecting a treatment training set 148 may be performed by generating classification algorithms by a processor. In an embodiment, a processor may receive diagnostic training data where the diagnostic training data includes a plurality of data entries containing comprehensive diagnoses containing urgent and non-urgent labels. Diagnostic training data may include any of the diagnostic training data as described above in reference to FIGS. 1-9. A processor generates a classification algorithm utilizing the diagnostic training data wherein the classification algorithm utilizes a plurality of user comprehensive diagnoses 144 as inputs and outputs a comprehensive diagnosis classification label for each of the plurality of user comprehensive diagnoses 144. For instance and without limitation, a comprehensive diagnosis such as Lyme Disease may contain an urgent classification label while a comprehensive diagnosis such as osteoarthritis may contain a non-urgent classification label. Classification algorithm includes any of the classification algorithms as described above in reference to FIGS. 1-9. A processor selects a treatment training set 148 as a function of a comprehensive diagnosis classification label for each of the plurality of user comprehensive diagnoses 144. In an embodiment, processor may match a comprehensive diagnosis classification label to a treatment training set 148 classifier label. For example, a comprehensive diagnosis classification label that contains an urgent label may be matched to a treatment training set 148 contained within training set database 152 that contains an urgent classifier label.

With continued reference to FIG. 10, at step 1035 a processor generates using a supervised machine-learning model a treatment 156 that outputs an ordered priority treatment list for each of the plurality of comprehensive diagnoses utilizing the selected treatment training set. Supervised machine-learning model includes any of the supervised machine-learning models as described above in reference to FIGS. 1-10. Treatment 156 includes any of the treatment 156 as described above in reference to FIGS. 1-10. Treatment 156 may include an algorithm, mathematical expression, series of calculations and the like as described above in more detail. Ordered priority treatment list includes any of the ordered priority treatment list as described above in reference to FIGS. 1-9. For instance and without limitation, a treatment 156 may be utilized to generate an ordered priority treatment list for a comprehensive diagnosis such as system Candida Albicans that includes an ordered priority treatment list that contains treatments that consist of first eliminating sugar from diet, second following a yeast free diet, third initiating a caprylic acid supplement regimen, fourth initiating a garlic supplement regimen, and fifth initiating a meditation practice.

With continued refence to FIG. 10, at step 1040 a processor evaluates one or more prioritized treatment facets contained within an ordered priority treatment list for a plurality of comprehensive diagnosis. Evaluating one or more prioritized treatment facets may include combining and eliminating one or more prioritized treatment facets relating to one or more comprehensive diagnoses. For example, evaluating one or more treatment facets may include examining a first comprehensive diagnosis such as broken ankle that contains a first treatment facet of ice, a second treatment facet of calcium supplementation, and a third treatment facet of rest. A processor may evaluate the treatment facets for the broken ankle as compared to treatment facets for a second comprehensive diagnosis such as myocardial infarction that contains a first treatment facet of following an anti-inflammatory diet and a second treatment facet that includes consuming a fish oil supplement. A processor may then evaluate all five total treatment facets to find any duplicates or any facets that may contraindicate one another and generate an ordered treatment plan that contains all five treatment facets in a prioritized order. A processor may determine how a treatment facet given a first priority for a first comprehensive diagnosis relates to a treatment facet given a first priority for a second comprehensive diagnosis. Evaluating may include determining which prioritized treatment facet for a plurality of comprehensive diagnoses will be placed in what order in treatment instruction set. This may be determined based on expert input such as what may be contained within expert knowledge database.

With continued reference to FIG. 10, evaluating one or more treatment facets may include correlating any user symptom data to one or more comprehensive diagnoses to determine the final order of treatment facets from one or more comprehensive diagnoses. A processor may retrieve an element of user symptom data from user data. An element of user symptom data may include any of the elements of user symptom data as described above in reference to FIGS. 1-9. A processor may correlate an element of user symptom data to a comprehensive diagnosis. Correlation may be performed utilizing any method as described above in reference to FIGS. 1-9. A processor may generate an ordered treatment instruction set 172 as a function of an element of user symptom data correlated to a comprehensive diagnosis. For example, a particular comprehensive diagnosis that has one or more elements of user symptom data correlated to it, may be utilized by a processor to generate an ordered treatment plan that contains one or more treatment facets related to the particular comprehensive diagnosis to be placed ahead of other treatment facets relating to other comprehensive diagnoses that do not contain any elements of user symptom data correlated to them.

With continued reference to FIG. 10, at step 1045 a processor generates a treatment instruction set 172 wherein the treatment instruction set 172 includes an ordered treatment plan containing one or more combined prioritized treatment facets for a plurality of comprehensive diagnoses. Generating an ordered treatment plan may be done utilizing any of the methods as described above in reference to FIGS. 1-10. Generating an ordered treatment plan may include receiving input from a comprehensive advisor. In an embodiment, a processor may receive a comprehensive input descriptor generated by a comprehensive advisor on a graphical user interface 146 located on the processor. Comprehensive input descriptor includes any of the comprehensive input descriptors as described above in reference to FIGS. 1-9. In an embodiment, comprehensive input descriptor may be stored locally such as within expert database 164. Comprehensive input descriptor includes an advisor interaction summary which may contain details describing a particular encounter between a user and a comprehensive advisor as described above in more detail. For example, an advisor interaction summary may include a description of progress a user may be making with a particular comprehensive diagnosis and may aid processor in selecting a first treatment facet to be placed in a first position within treatment instruction set. In yet another non-limiting example, a particular advisor interaction summary may contain a description from a physical examination of a user by a comprehensive advisor and may dictate what treatment facet needs to be addressed first.

With continued reference to FIG. 10, generating a treatment instruction set 172 may include utilizing user inputs relating to short term and long-term target indicators to select a first treatment facet and a last treatment facet in an ordered priority. A processor may retrieve a user diagnostic factor input from user database wherein the user diagnostic factor input includes a long-term target indicator and short-term target indicator. Diagnostic factor input may include any of the diagnostic factor input as described above in more detail in reference to FIGS. 1-10. A processor may generate a loss function utilizing the user diagnostic factor input and minimize the loss function. This may be performed utilizing any of the methods as described above in reference to FIGS. 1-10. A processor may then generate an ordered treatment plan as a function of minimizing the loss function. For example, a particular treatment facet may be placed in last place within treatment instruction set 17 2that fulfills a particular long-term target indicator while a particular treatment facet that fulfills a user's short-term target indicator may be placed in first place within treatment instruction set. Treatment instruction set 172 may also be generated based on calculating a comprehensive diagnosis impact score. Comprehensive diagnosis impact score may include factor scores generated by users, comprehensive advisors, and expert input that may help dictate placement of treatment facets in a particular order within treatment instruction set 172 based on factors that include how difficult a particular treatment facet may be to implement, what types of resources and how many resources a user may have to use to fulfill a particular treatment facet, as well as how much effort a user may be willing to complete a particular treatment facet. Factors may be given numerical scores as described above in more detail in reference to FIGS. 1-9.

With continued reference to FIG. 10, generating treatment instruction set 172 may include placing treatment facets in a particular order as a function of a treatment facets priority for a particular comprehensive diagnosis as well as a treatment facets priority as compared to other treatment facets within treatment instruction set. For example, generating an ordered treatment plan may include selecting a first treatment facet as a function of a first priority treatment, selecting a second treatment facet as a function of the first treatment facet, and selecting a third treatment facet as a function of the second treatment facet. In yet another non-limiting example, a first treatment facet may be selected as a function of a first priority treatment, a second treatment facet may be selected as a function of the first priority treatment, and a third treatment facet may be selected as a function of the second priority treatment.

With continued reference to FIG. 10, at step 1050 a processor displays a treatment instruction set 172 on a graphical user interface 146 located on a processor. A treatment instruction set 172 may be displayed utilizing any methods as described herein. In an embodiment, a treatment instruction set 172 may be displayed on a graphical user interface 146 so that a comprehensive advisor can select a particular treatment facet to obtain more detailed information about that particular treatment facet and what treatment for that particular treatment facet includes. For example, a particular treatment facet such as “initiating a multi-vitamin supplement regimen” may provide more detailed information such as potential recommended supplements for a user based on a user's age, sex, medical history, concurrent comprehensive diagnoses, as well as other medications and supplements that a user may be taking. In yet another non-limiting example, a particular facet such as “start treatment with rifaximin” may contain additional information for the comprehensive diagnosis such as the ideal dose for a user based on a user's age, sex, height, weight, other medication conditions, health history, drug allergies and the like.

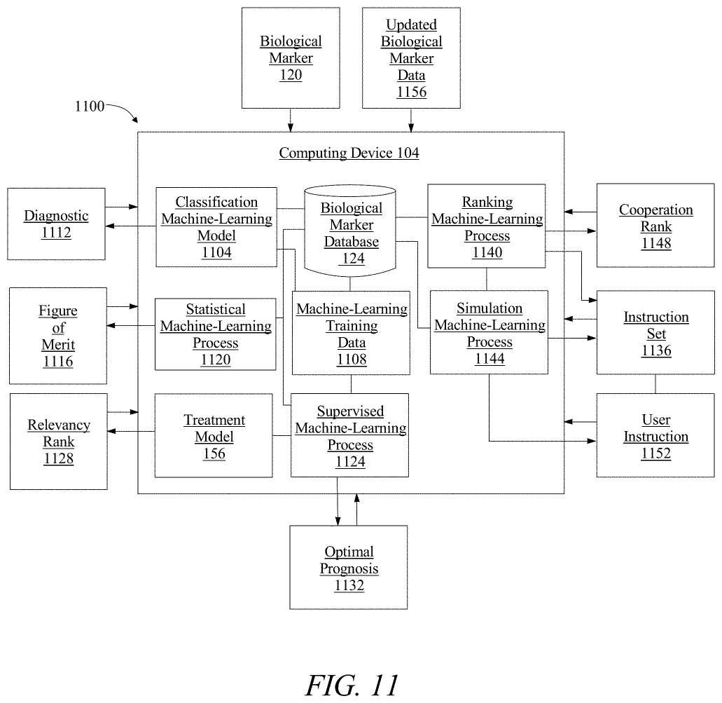

Referring now to FIG. 11, a comprehensive prognosis system 1100 for prioritizing comprehensive prognoses and generating an associated treatment instruction set is illustrated. Comprehensive prognosis system 1100 comprises a computing device 104, wherein a computing device 104 may be configured to receive at least a user biological marker 120, as described above. Computing device 104 may include a computing device, as described above, and computing device 104 may include a graphics processing unit (GPU), or any other computing device containing a processing capability. Biological marker 120 may include user wearable device data and/or analysis of such data, as described above, that is data that contains at least a marker of disease. In non-limiting illustrative examples, wearable device data may include without limitation accelerometer data, pedometer data, gyroscope data, electrocardiography (ECG) data, electrooculography (EOG) data, bioimpedance data, blood pressure and heart rate monitoring, oxygenation data, biosensors, fitness trackers, force monitors, and the like, as described above. Computing device 104 may receive wearable device data containing biological marker 120 and store and/or retrieve data from a biological marker database 124, such as in a sensor data table 412.

Continuing in reference to FIG. 11, comprehensive prognosis system 1100 receiving biological marker 120 may include using a wearable device to collect biological marker 120 data of a user and classifying, using the classification machine-learning model, the user biological marker to a diagnostic profile based on the collected wearable device data. A classification machine-learning model may be any machine-learning process for deriving models, as described above. A classification machine-learning model 1104 may retrieve a biological marker classification label 136 and the associated data to train the model in determining a diagnostic associated with a user depending on comprehensive diagnoses 144, treatments, and the like, that may be associated with alike users. Classification machine-learning model 1104 may accept an input of a plurality of elements of biological marker 120 data and generate an output that is at least a diagnostic as it relates to the biological markers data, as described in further detail below. For instance in non-limiting illustrative examples, classification machine-learning model 1104 may accept biological marker 120 data that includes blood panel results, including identities and concentrations of blood proteins and enzymes, cytokines, LDL/HDL cholesterol, blood sugar, iron and other trace elements, vitamins, minerals, the like, and generate an output based on the training data that relates the biological marker 120 data to symptoms, diseases, conditions, or the like, that may pertain to each of the elements of data in the biological marker 120 data, such as anemia, diabetes, physical exertion, liver disease, viral and bacterial infection, excessive thirst, and the like.

Continuing in reference to FIG. 11, computing device 104 may select a prognosis, wherein selecting the prognosis using a computing device 104 may include training a classification machine-learning model 1104 using machine-learning training data 1108 that corresponds to the user biological marker 120 and an associated diagnostic relevancy from a biological marker database 124 as a function of the user biological marker. Classification machine-learning model 1104 may be trained using machine-learning training data 1108, as described above. Machine-learning training data 1108 may include any user biological marker 120, wearable device data, and/or analysis of the data. Machine-learning training data 1108 may include model, functions, matrices, functions, vectors, heuristics, and/or any other determinations, relationships, and the like, made from a machine-learning model as training data to train a classification machine-learning model 1104 for purposes described herein. Classification machine-learning model 1104 trained with machine-learning training data 1108 may match an associated diagnostic to the model trained with at least a user biological marker 120.

Continuing in reference to FIG. 11, computing device may determine a diagnostic using the at least a user biological marker 120. A “diagnostic,” as used in this disclosure, is a combination of any symptoms, diseases, conditions, diagnoses, and/or cognitive, biological, and/or physiological deteriorations that are present and/or determined from at least a user biological marker 120 by a machine-learning process, including any user lifestyle data such as current diet, nutrient deficiencies, exercise frequency, duration, and type, and sleep quality factors. For instance in non-limiting illustrative examples, a diagnostic from user biological markers 120 from blood chemistry may indicate Type-II diabetes, despite the user having been and/or having not been diagnosed with Type-II diabetes yet; a diagnostic 1112 may be a series of symptoms that a user is expected to experience associated with Type-II diabetes, such as increased thirst, frequent urination, hunger, fatigue, and/or blurred vision. A diagnostic 1112 may contain a biological marker classification label 136 and/or a comprehensive diagnosis 144, as described above. A diagnostic 1112 may include symptoms that may be addressed by user lifestyle changes, such as adhering to a specific diet, exercise regimen, and the like. A diagnostic 1112 may be addressed by a treatment, wherein a treatment may be a prescription administered by a medical professional, or the like, and/or a treatment may be a proscribed user lifestyle change that system 100 may assist user with pursuing prior to a disease, condition, and/or deterioration appears. Classification machine-learning model 1104 may classify the user biological marker to a diagnostic profile, or a plurality of potential diagnostics, based on wearable device data, and potentially differentiate between potential diagnostics, as described in further detail below.

In further non-limiting illustrative examples, a distinction between a comprehensive diagnosis 144 and a diagnostic 1112 is that a comprehensive diagnosis 144 may be a condition or disease that may be observed directly or not observed directly by the user, physician, or the like, but a diagnostic 1112 may include a comprehensive diagnosis 144 and any symptoms or deteriorations observed by the user and/or physician associated with a comprehensive diagnosis 144. This distinction is important in that some treatments may be provided directly for dealing with symptoms and/or how the body deteriorates rather than addressing the etiology of a diagnosis. Alternatively or additionally, lifestyle changes such as exercise regimes, dietary changes, and the like, implemented as treatments by a user may not directly address a comprehensive diagnosis 144, but may greatly affect a diagnostic 1112. In a non-limiting exemplary embodiment, a comprehensive diagnosis 144 may be a diagnosis of cancer, wherein the user has may or may not have current symptoms, wherein a diagnostic 112 would include the diagnosis of cancer and any symptoms, additional conditions, and how a body deteriorates that may be currently experienced or may manifest in the future according to a biological marker 120 that may have been used to determine the original diagnosis. In such an example, the comprehensive diagnosis 144 may not change over the course of the disease, wherein the cancer diagnosis is kept so long as the cancer is present, but the diagnostic 1112 (based on the same biological markers or not) may change as symptoms appear such as nausea, rash, lethargy, difficult staying asleep, how the body changes such as hair loss and brittle nails, and user lifestyle changes such as altered diet and altered physical fitness. If this were the case, for instance and without limitation, a treatment may include methods adoptable by the user to change to a ketogenic diet and adopt a physical rehabilitation program to improve quality-or-life and address symptoms in the diagnostic 1112, but may not have any effect on the comprehensive diagnosis 144.

Continuing in reference to FIG. 11, computing device 104 may rank the at least a diagnostic 1112 using the trained classification machine-learning model 1104, wherein ranking further comprises using a statistical machine-learning process to determine a figure of merit of a diagnostic 1112 matching the user biological marker 102. A “figure of merit,” as used in this disclosure, is a numerical value, percentile, percentage, function, matrix, vector, or the like, that represents or otherwise summarizes a statistical analysis of accuracy of the diagnostic 1112 as it may match, or be associated with, a biological marker 120 of a user. In non-limiting illustrative examples, a diagnostic 1112 may have a relatively low figure of merit 1116 from a single biological marker 120, but with more biological marker 120 data provided, the figure of merit 1116 may indicate increased agreement between diagnostic and biological markers 120. In such an example, the figure of merit 1116 may signal to a medical professional, caretaker, user, or any other, the accuracy of the diagnostic 1112 associated with the data pertaining to a user.

A statistical machine-learning process used for ranking a plurality of diagnostics 1112 may include any machine-learning algorithm used by a supervised machine-learning process, as described above. Alternatively and/or additionally, a statistical machine-learning process 1120 may include a supervised and/or unsupervised machine-learning process. Alternatively or additionally, a statistical machine-learning process 1120 may be an online learning process, wherein an online machine learning process utilizes a method that, as data becomes available in a sequential order, the process is used to generate updated values that represent the best predictor for future data at each step. In supervised and/or unsupervised learning online machine learning processes, a function of ƒ: X→Y is to be learned, wherein Xis thought of as a space of inputs and Y as a space of outputs, and a prediction on the instances that may be drawn from a joint probability distribution, p(x, y), on X×Y. The machine-learning algorithm may never know the true distribution of p(x, y) over instances. Instead, the machine-learning algorithm may access a training set of examples (x₁, y₁), . . . , (xₙ, yₙ). In this instance, a loss function may be given as V : Y×Y→ [symbol] , such that V(ƒ(x), y) measures the difference between the predicted value ƒ(x) and the true value, y. The ideal goal is to select a function ƒ∈ [symbol] , where [symbol] is a space of functions called a ‘hypothesis space’, so that some notion of total loss is minimized. Depending on the type of model, such as a statistical model and/or an adversarial model, the statistical machine-learning process 1120 can devise different notions of loss, leading to different variations of the learning algorithms, and different variations of output values.

Statistical online machine learning process may use, for instance and without limitation, an empirical risk minimization, regularized empirical risk minimization, Tikhonov regularization, and the like, wherein the data in the training sample are assumed to have been drawn from a true distribution and the objective is to minimize the expected “risk” of a predicted value from the training sample, given some relationship between the predicted values and the training sample. In such examples, the choice of loss function may in minimization may be selected from a plurality of machine-learning algorithms such as regularized least squares analysis, linear least squares, stochastic gradient descent, incremental stochastic gradient descent, batch learning, online convex optimization, support vector machines, and/or kernel methods. In a non-limiting illustrative example, any of the above algorithms may be performed by a statistical machine-learning process 1120 wherein an input of a first set of values described by a plurality of diagnostics 1112 and/or plurality of figure of merits 1120 are input, and a set of information may be retrieved that describes a ranking, relationship, correlation, or other heuristic that relates a second predicted set of values related in some manner to the first set of values. In such an example, a statistical machine-learning process 1120 may output a series of values that correspond to a likelihood of a plurality of diagnostics 1112 to match biological markers 120 ranked as a function of their associated figure of merit 1116. Each figure of merit 1116 may be a predicted value that is statistically associated with a first set of data, such as a plurality of diagnostics 1112 used as inputs for the algorithm.

Statistical machine-learning process 1120 may be a reinforcement learning process, such as a Markov decision process (MDB), or the like. Reinforcement learning processes may be modeled as discrete-time stochastic control processes, wherein there exists a set of environment and agent states, S, a set of actions, A, of the agent, wherein Pₐ(s, s′)=Pr(sₜ₊₁=s′|sₜ=s, αₜ=α) is the probability of transition, at time t, from state s to state s′ under action, a. In such an example, the rules are often stochastic, in that numerical values may be randomly selected. It is assumed the agent may observe the current environment, or partially observe, wherein the set of actions is restricted, for instance, a numerical value may not be reduced below 0, for instance if the current value of the agent is 3, and the state transition reduces the value by 4, the transition will not be allowed, and an associated probability determined for the transition will reflect that. In non-limiting illustrative examples, a reinforcement learning process may be utilized to determine the transition state of a particular set of values described in a plurality of diagnostics 1112 to generate final values that correspond to a ranking of a plurality of diagnostics 1112 based on a plurality of figure of merits 1120, wherein the transition is dictated by some predicted relationship between the first and second function. Likewise, in non-limiting illustrative examples, a reinforcement learning process may be used to determine predicted values of rankings based on figure of merit 1116 over long periods of time by optimizing a new state, sₜ₊₁, with a reward, rₜ₊ᵢ, associated with the transition. The goal of the reinforcement learning agent is to maximize the reward. The agent can choose any action, randomly or not, as a function of the history of values. In non-limiting illustrative examples, this may be performed to generate optimal predicted figure of merit 1116 and associated rankings based wherein the plurality of diagnostics 1112 may not directly inform what the values should be, but be used to optimally predict values for a long period, such as determining the accuracy of a diagnosis, of a plurality of diagnoses, for a user over the life time of the user.

Continuing in reference to FIG. 11, computing device 104 may select a prognosis based on the ranking based on the figure of merit 1116 of the diagnostic 1112. A “prognosis,” as used in this disclosure, refers to a predicted, expected, calculated, simulated, theoretical, or otherwise determined outcome from selecting a diagnostic and employing a treatment to the diagnostic using a machine-learning process. In non-limiting illustrative examples, a treatment 156 may be a course of action prescribed to a user in addressing a symptom, diagnosis, condition, or the like, that can result in a prognosis, wherein the prognosis is a determined outcome from using the treatment 156. In further non-limiting illustrative examples, a treatment 156 may be a course of action proscribed to a user, wherein the treatment 156 is to prevent a potential diagnostic from manifesting in the user biological markers 120.

Continuing in reference to FIG. 11, computing device 104 may generate at least a treatment for a diagnostic 1112, wherein generating at least a treatment may include using a supervised machine-learning process. A supervised machine-learning process may be any machine-learning process, as described above. Supervised machine-learning process 1124 may accept an input of a diagnostic selected based upon a ranking based upon a figure of merit 1116, as described above. Supervised machine-learning process 1124 may then query a database, such as an online research repository, expert submission, medical database, nursing textbook, diagnostic manual, or the like, to retrieve at least a treatment 156 for addressing the diagnostic 1112.

Continuing in reference to FIG. 11, computing device 104 generating the at least a treatment 156 may include generating a supervised machine-learning process 1124 to query a database for a treatment 156, and determine a relevancy rank for a treatment 156 in addressing a diagnostic 1112, wherein a relevancy rank relates to the ability of the treatment 156 to result in an optimal prognosis. A “relevancy rank,” as used in this disclosure, is a numerical value, percentile, percentage, function, matrix, vector, or the like, that represents or otherwise summarizes a statistical analysis of accuracy of a treatment 156 to address a diagnostic 1112. In non-limiting illustrative examples, a relevancy rank 1128 may be determined by a supervised machine-learning process 1124, and/or by a statistical machine-learning process as described above, by retrieving patient prognoses relating to a treatment 156 of an adenoidectomy in addressing obstructive sleep apnea (OSA) and snoring. In further non-limiting illustrative examples, a relevancy rank 1128 may be a numerical value that relates to the percent of patients that experience a reduction in their OSA and/or snoring after electing to receive the surgery.

An ‘optimal prognosis,” as used in this disclosure, is a prognosis associated with a treatment that results in the most optimal outcome for the treatment in addressing a diagnostic 1112. An optimal outcome may include an outcome that results in a favorable result for a user, such as for example the suppression of disease symptoms, reversal of disease progression, elimination of disease, remission of disease and the like. In such an example as above, the optimal prognosis 1132 for such a treatment may include the recovery from any symptoms, medications, lengths of time, and the like, associated with the surgery, and may or may not reflect an ‘optimal prognosis’ due to whether the treatment is associated with the highest accuracy relevancy rank 1128. In further non-limiting illustrative examples, an optimal prognosis 1132 may be for a first treatment of a plurality of treatments, wherein a diagnostic responds best to the first treatment of the plurality of treatments.

Continuing in reference to FIG. 11, computing device 104 may rank, using the supervised machine-learning process 1124, an instruction set 1136 of the at least a treatment 156, wherein ranking an instruction set 1136 may include uses a ranking machine-learning process to generate a ranked list of instructions for implementing the treatment. An “instruction set,” as used in this disclosure is a series of steps, performed by a medical professional, caretaker, and/or the user, that are associated with implementing a treatment 156. Instruction set 1136 may consist of steps that may correspond to more than a single treatment, for instance and without limitation, steps that correspond to a procedure carried out by a medical professional and a lifestyle change performed by a user. Ranking instructions of an instruction set 1136 may include using a ranking machine-learning process 1140, wherein ranking machine-learning process 1140 ranks instructions based on a chronological ordering that results in the optimal prognosis 1132. A ranking machine-learning process 1140 may be an algorithm, or series of algorithms, that sorts inputs and ranks outputs using a numerical value, or the like, as described above. A ranking machine-learning process 1140 may be a machine-learning model, process, or the like, as described above, that accepts an instruction set 1136 and/or a plurality of instruction sets 1140 to generate an output that is a series of individual instructions ordered for a user to follow. In non-limiting illustrative examples, ranking machine-learning process 140 may accept an input of a series of steps relating to a treatment and generate an output that is a ranked series of instructions associated with the optimal prognosis for that treatment.

Continuing in reference to FIG. 11, computing device 104 ranking an instruction set 1136 using a ranking machine-learning process 1140 may include ranking instructions based on a chronological ordering that results in in an optimal prognosis 1132. An optimal prognosis 1132 may be a predicted outcome associated with a treatment 156, as described above. Such a treatment 156 may represent the selected treatment 156 for a diagnostic 1112, as described above.

Continuing in reference to FIG. 11, computing device 104 selecting a prognosis includes simulating the at least an instruction set 1144 using a simulation machine-learning process, wherein simulating using the simulation machine-learning process may include generates a prognosis that would result from implementing each instruction set 1136. A simulation machine-learning process may be a probabilistic technique such as simulated annealing algorithm, among other randomized algorithms for performing simulations, such as Las Vegas algorithm, Monte Carlo algorithms, and the like.

A simulation machine-learning process 1144 may make use of statistical machine-learning process 1116, as described above. A simulation machine-learning process 1144 may perform a simulation by randomly perturbing parameters, such as medication dosages, nutrition, and the like, and determine the effect on a diagnostic (such as a symptom and/or diagnosis), to determine which parameters result in an optimal prognosis 1132, or optimal outcome.

In non-limiting illustrative examples, a simulation machine-learning process 1144 may be a Monte Carlo algorithm, or similar simulation machine-learning process as described above. A Monte Carlo simulation is a mathematical technique that may generate variables, numerical values, and the like, for modeling risk, outcomes, uncertainty, etc., of a certain system using a stochastic simulation process. Monte Carlo simulations may encompass a range of algorithms and mathematical analysis techniques such as Markov Model Monte Carlo (MMMC) simulations, McKean-Vlasov processes, Monte Carlo localization, among other probabilistic stochastic heuristics that randomly select numerical parameters from within a defined set of parameters and calculate an outcome for all selected parameters. A Monte Carlo simulation may generate a series of numerical values represented by traces, curves, functions, and the like, wherein each function may represent a sufficiently good solution and/or outcome to an optimization problem, wherein the solution may be represented by a polar coordinate, vector, function, or the like, that represents, for instance and without limitation, how a diagnostic 1112 improves from implementing an instruction set 1136, wherein the order, magnitude, timing, and the like, of the individual instructions may be perturbed for each simulation. An “instruction set,” as used in this disclosure is a series of incremental steps, for implementing and/or following a treatment. For instance and without limitation, such steps may be instructions involved in taking a medication, instructions to nursing a soft-tissue injury, or implementing a ketogenic diet. Each generated treatment 156 may have an associated instruction set 1136, wherein each instruction set 1136 from a simulation may have associated with it changes in prognosis. Simulated parameters that result in an optimal prognosis 1132 may reflect an outcome with an updated, or otherwise modified, instruction set 1136, wherein this modified instruction set 1136 may include new instructions for instance new user actions, different timing of instructions, or the like. Persons skilled in the art, upon review of this disclosure in its entirety, will be aware of the various simulation machine-learning processes that may be performed by a computing device 104 to sample parameters associated with a treatment and calculate an associated prognosis.

Continuing in reference to FIG. 11, computing device 104 performing a simulation machine-learning process 1144 to generate a simulated user state may include sampling at least a biological parameter associated with implementing an instruction set 1136, wherein the simulation perturbs the biological parameter. A “biological parameter,” as used in this disclosure is any element of qualitative and/or quantitative data present in biological marker 120 data of a user, including any diagnostic data, treatment parameters such as doses, frequencies, exercise regimens, diets, and the like. For instance in non-limiting illustrative examples, a simulation may determine a large degree of curves tracing user prognoses regarding recovery from cancer, wherein the instruction set 1136 for a treatment 156 may include dozens of a plurality of medical interventions such as surgeries, chemotherapy, XRT treatments, and cancer screening, as well as lifestyle changes, such as quitting tobacco products, changing diet, improving sleep quality, and the like; wherein each curve may be a series of numerical values of 5-year survival, overall health score, likelihood of recovery, and the like, plotted as a function of time for a wide variety of biological parameters, sampled in finite increments. And thus, each biological parameter may have an estimated prognosis (such as a 5-year survival rate) associated therewith. In a non-limiting illustrative example, each category (treatment, lifestyle change, etc.) may have a simulation performed for each biological parameter that is associated with it (timing of treatment, type of diet changed to, hours of REM sleep, nicotine dose reduction over time, etc.); wherein all biological parameters are held constant, and only one is sampled at a time.

Continuing in reference to FIG. 11, computing device 104 performing a simulation machine-learning process 1144 to generate a simulated user state may include determining an effect of the simulated biological parameter, wherein determining an effect is determining how at least an element of biological marker data may change as a function of the simulated biological parameter. Simulation machine-learning process 1144 may sample biological parameters associated with a treatment 156 and determine an output that is a numerical value describing how a prognosis affects the diagnostic 1112, and thus the at least a biological marker 120 associated with the diagnostic 1112. For instance in non-limiting illustrative examples, simulation machine-learning process 1144 may output a prognosis that contains a numerical value that is a percent decrease in a biological marker that was associated with a diagnostic 1112 that a treatment was expected to address.

Continuing in reference to FIG. 11, computing device 104 performing a simulation machine-learning process 1144 to generate a simulated user state may include identifying at least a simulated biological parameter that results in a ranked treatment 156 that results in an optimal prognosis 1132 from implementing the instruction set 1136. Simulation machine-learning process 1144 may simulate a plurality of treatments 156 and rank a plurality of treatments 156 using a ranking algorithm, as described above, for instance and without limitation based on the simulated prognosis. An instruction set 1136 may be selected pursuant of a treatment 156 that resulted in the most optimal prognosis.

Continuing in reference to FIG. 11, computing device 104 ranking an instruction set 1136 may include modifying an instruction set 1136 to reflect at least a simulated biological parameter. A plurality of instruction sets 1140 may be ranked, for instance and without limitation, by a difficulty factor, prognosis, and/or any other parameters described herein. An instruction set 1136 may be modified from the original instructions to reflect any changes in parameters that were output by a simulation. For instance in non-limiting illustrative examples, a simulation may determine that an instruction set 1136 may have scheduling of treatments moved, or perhaps a medication dose increased due to medical history, among other parameters.

Continuing in reference to FIG. 11, computing device 104 ranking a prognosis using the trained classification machine-learning model 1104 may include ranking a plurality of diagnoses as a function of a ranking machine-learning process 1140 and the figure of merit 1116 of each diagnosis. Computing device 104 may rank a plurality of diagnosis, such as user comprehensive diagnoses 144, pertaining to a plurality of diagnostics 1112 using ranking machine-learning process 1140, as described above. Ranking diagnoses may refer to ranking based on the likelihood the diagnoses match at least a biological marker 120 based on calculated accuracy as described by a figure of merit; likewise ranking diagnoses may refer to ranking based on survival, treatability, prognosis, or any other parameters that were, perturbed, tested or otherwise determined as an output by a simulation, as described above. In non-limiting illustrative examples, computing device 104 may retrieve a plurality of diagnoses, such as user comprehensive diagnoses 144 from a database, such as a user database 300 and/or a biological marker database 124, and simulate at least a prognosis for each of a plurality of diagnoses, wherein each diagnosis will have an output associated with its likelihood to match a biological marker 120 and an outcome associated with treatment of the diagnosis.

Continuing in reference to FIG. 11, computing device 104 selecting a prognosis may determine a rank for each prognosis generated. In non-limiting illustrative examples, computing device 104 may use a ranking machine-learning process 1140, as described above, to determining a rank which may refer to a numerical ranking of each prognosis based on some criterion, such as survivability, treatment options, time of recovery, and the like. In such an example, computing device 104 may select a prognosis with an associated instruction set 1136 for a treatment 156 of a diagnostic 1112 based on such a ranking.

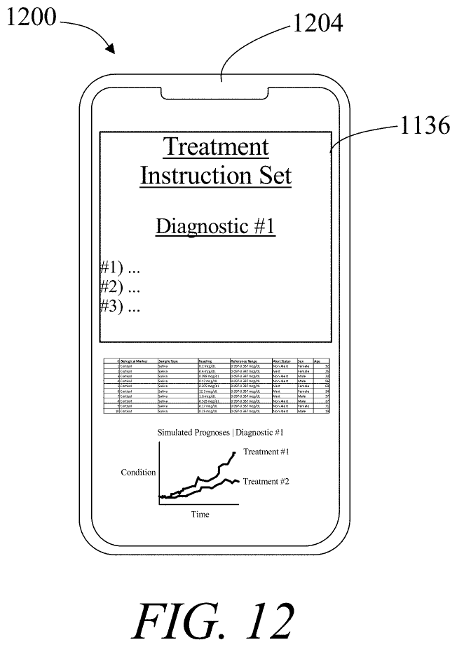

Referring now to FIG. 12, a non-limiting exemplary embodiment 1200 of a user device 1204 is illustrated. Computing device 104 may display, using a graphical user interface, the treatment instruction set 1136 via a user device 1204. A “user device,” as used in this disclosure is any smartphone, laptop, tablet, computing device, or the like, that may display via a graphical user interface (GUI) text, graphics, or any other information. Persons skilled in the art, upon review of this disclosure in its entirety, will be aware of the various ways in which a graphical user interface may be implemented for the purposes described herein, and the various devices which may be used as user devices.

Referring back to FIG. 11, computing device 104 is configured to direct a user to update user data after a first instruction set is provided, wherein directing may include setting notifications to remind a user to follow-up, determining a cooperation rank, wherein the cooperation rank 1148 relates a user rank with the level of cooperation and completion of treatment instructions, and providing, via a user device 1204, a cooperation rank 1148 that may increase as a user provides user data. A “cooperation rank,” as used in this disclosure is a rank, score, or other metric, which may be represented by a numerical value, percentile rank, or the like, that corresponds to a user's progress in a treatment and feedback on how a user is addressing a diagnostic. A cooperation rank may be designed to improve user feedback, follow through, and cooperation with treatment instructions. In non-limiting illustrative examples, a cooperation rank 1148 may refer to a user's percentile score of recovery for following a treatment in addressing a diagnostic, wherein the cooperation rank 1148 may be a numerical value that states a user is recovering at a particular percentile among those affected by the diagnostic. Cooperation rank 1148 may be determined by a computing device 104 by using a machine-learning process, as described above, to compare a user's progress in treatment and/or severity of biological markers 120 to biological markers and/or progress in treatment to others, for instance and without limitation, as retrieved from an online database by a query. A cooperation rank 1148 of a user may increase with successive user data updates; this may include biological markers, selecting treatments that have been completed, updating wearable device data, and the like. Computing device 104 may direct a user to update user data, such as biological markers, selecting treatments that have been completed, updating wearable device data, and the like, by sending notifications to a user device. Notifications may be alarms, messages, and the like, that prompt, or otherwise direct a user to update user data to system 100. Cooperation rank 1148 may be provided to a user via a user device 1204, as described above.

Referring now to FIG. 11, computing device 104 providing a user cooperation rank 1148 may include providing a user instruction 1152, wherein the user cooperation rank may increase as a function of the user completing an instruction. A “user instruction,” as used in this disclosure is an instruction of an instruction set 1136 that may be intended directly for a user to complete, as opposed to a physician, medical professional, physical trainer, caretaker, or the like. Completing a user instruction 1152 may refer to a user completing an instruction that is directed to the user regarding addressing a diagnostic 1112, providing a biological marker 120, implementing a treatment 156, following an instruction set 1136, and/or obtaining an optimal prognosis 1132. In non-limiting illustrative examples, a user instruction 1152 may be a prompt to update user data, for instance in directing a user to update biological marker data such as updating blood chemistry results after taking an iron supplement, wherein a cooperation rank 1148 of a user may increase when the user completes the user instruction 1152 and provides the updated biological marker data.

Continuing in reference to FIG. 11, computing device 104 may receive updated biological marker data 1156, wherein updated biological marker data 1156 is user data provided more recent in time than a first instruction set 1136 was displayed, compute, using the classification machine-learning model 1104 and the updated biological marker data 1156, a numerical difference between a first figure of merit 1116 of the first prognosis with an updated figure of merit 1116, and determine if the numerical difference between the first figure of merit 1116 and the updated figure of merit 1116 results in an improved prognosis. An improved prognosis may refer to any indication that the outcome has improved from when a diagnostic 1112 was first selected for a user based on at least a biological marker 120 that was provided. Update biological marker data 1156 may refer to at least a biological marker 120 which may be used to calculate a figure of merit 1116 for a diagnostic, as described above, to determine if a diagnostic 1112 may have been partly treated, completely resolved, or the like. Comprehensive prognosis system 1100 may use updated biological marker data 1156 to determine if a treatment 156 and corresponding instruction set 1136 had an intended effect that was described in an optimal prognosis 1132.

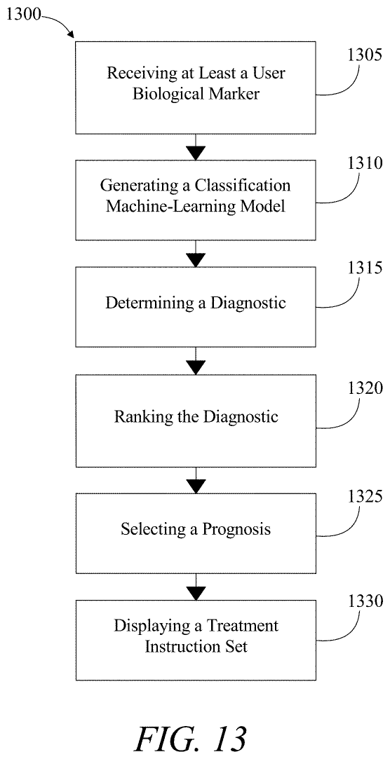

Referring now to FIG. 13, a non-limiting exemplary embodiment of a method 1300 for prioritizing comprehensive prognoses and generating an associated treatment instruction set is illustrated. At step 1305, computing device 104 receives at least a user biological marker 120. User biological marker 120 may include wearable device data. User biological marker 120 may include using a wearable device to collect biological marker data of a user, and classifying, using the classification machine-learning model 1104, the user biological marker 120 to a diagnostic 1112 profile based on the collected wearable device data; this may be implemented without limitation at described above in FIG. 1-12.

With continued reference to FIG. 13, at step 1310, computing device 104 may generate a classification machine-learning model 1104 using a machine-learning training data 1108 wherein the machine-learning training data 1108 contains a plurality of data entries containing biological markers 120 as inputs corresponding to associated diagnostics 1112 as outputs; this may be implemented without limitation at described above in FIG. 1-12.

With continued reference to FIG. 13, at step 1315, computing device 104 may determine a diagnostic 1112 using the at least a user biological marker 120, the classification machine-learning model, and the machine-learning training data 1108; this may be implemented without limitation at described above in FIG. 1-12.

With continued reference to FIG. 13, at step 1320, computing device 104 may rank the diagnostic 112, wherein ranking may include using a statistical machine-learning process 1120 to determine a figure of merit 1116 of a diagnostic 1112 matching the user biological marker 120; this may be implemented without limitation at described above in FIG. 1-12.

With continued reference to FIG. 13, at step 1325, computing device 104 may select a prognosis as a function of the figure of merit 1116, wherein selecting the prognosis may include generating at least a treatment 156 using a supervised machine-learning process 1124, wherein generating a treatment 156 using a supervised machine-learning process 1124 may include determining an instruction set 1136 for the treatment 156, simulating the at least an instruction set 1136 using a simulation machine-learning process 1144, wherein simulating using the simulation machine-learning process 1144 may include performing a simulation machine-learning process 1144, wherein the simulation machine-learning process 1144 generates a prognosis that would result from implementing each instruction set 1136, determining a rank for each prognosis, and providing the instruction set that results in the optimal prognosis 1132. Generating at least a treatment 156 may include retrieving a treatment 156 using a supervised machine-learning process 1124 to query a database for a treatment 156, wherein querying is performed as a function of preferences located in user data, determining a relevancy rank 1128 for the treatment 156 in addressing a diagnostic 1112, wherein the relevancy rank 1128 relates to the ability of the treatment 156 to result in an optimal prognosis 1132, and providing an instruction set 1136 for the treatment 156, wherein an instruction set 1136 is a series of instructions a user may perform to address a diagnostic 1112. Generating at least a treatment 156 may include ranking the instructions of an instruction set 1136 using a ranking machine-learning process 1140, wherein the ranking machine-learning process 1140 ranks instructions based on a chronological ordering that results in in an optimal prognosis 1132 for the treatment 156. Performing the simulation machine-learning process 1144 to generate the simulated user state may include sampling at least a parameter in the biological marker 120 associated with implementing an instruction set 1136, wherein the simulation perturbs the biological parameter, determining an effect of the simulated biological parameter, wherein determining may include calculating how at least an element of biological marker 120 data may change as a function of the simulated biological parameter, and identifying at least a simulated biological parameter that results in a ranked treatment that results in an optimal prognosis 1132 from implementing the instruction set 1136. Ranking may include modifying the instruction set 1136 to reflect at least a simulated biological parameter. Ranking the prognosis using the trained classification machine-learning model 1104 may include ranking a plurality of diagnoses as a function of a ranking machine-learning process 1140 and the figure of merit 1116 of each diagnosis; this may be implemented without limitation at described above in FIG. 1-12.

With continued reference to FIG. 13, at step 1330, computing device 104 may display, using a graphical user interface, the treatment instruction set 1136. Computing device 104 is further configured to direct the user to update user data as a function of a first instruction set 1136, wherein directing may include displaying a notification to remind the user to follow-up, determining a cooperation rank 1148, wherein the cooperation rank 1148 relates a user rank with the level of cooperation and completion of treatment instructions, and providing, via the user device, the cooperation rank 1148. User cooperation rank 1148 may increase as a function of the user completing an instruction. Computing device 104 is further configured to receive updated biological marker 120 data, where updated biological marker 120 data is more recent in time than when a first user data, compute, using the classification machine-learning model 1104 and the updated biological marker data 1156, a numerical difference between a first figure of merit of the first prognosis with an updated figure of merit, and determine a prognosis state as a function of a numerical difference between the first figure of merit and the updated figure of merit.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

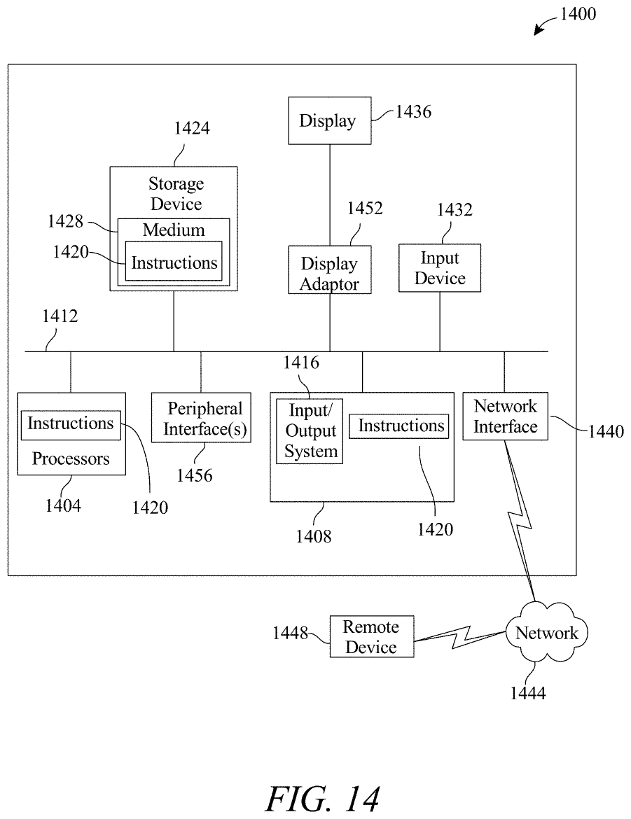

FIG. 14 shows a diagrammatic representation of one embodiment of a computing device in the exemplary form of a computer system 1400 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1400 includes a processor 1404 and a memory 1408 that communicate with each other, and with other components, via a bus 1412. Bus 1412 may include any of several types of bus structures including, but not limited to, a memory bus, a memory controller, a peripheral bus, a local bus, and any combinations thereof, using any of a variety of bus architectures.

Memory 1408 may include various components (e.g., machine-readable media) including, but not limited to, a random access memory component, a read only component, and any combinations thereof. In one example, a basic input/output system 1416 (BIOS), including basic routines that help to transfer information between elements within computer system 1400, such as during start-up, may be stored in memory 1408. Memory 1408 may also include (e.g., stored on one or more machine-readable media) instructions (e.g., software) 1420 embodying any one or more of the aspects and/or methodologies of the present disclosure. In another example, memory 1408 may further include any number of program modules including, but not limited to, an operating system, one or more application programs, other program modules, program data, and any combinations thereof.

Computer system 1400 may also include a storage device 1424. Examples of a storage device (e.g., storage device 1424) include, but are not limited to, a hard disk drive, a magnetic disk drive, an optical disc drive in combination with an optical medium, a solid-state memory device, and any combinations thereof. Storage device 1424 may be connected to bus 1412 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1424 (or one or more components thereof) may be removably interfaced with computer system 1400 (e.g., via an external port connector (not shown)). Particularly, storage device 1424 and an associated machine-readable medium 1428 may provide nonvolatile and/or volatile storage of machine-readable instructions, data structures, program modules, and/or other data for computer system 1400. In one example, software 1420 may reside, completely or partially, within machine-readable medium 1428. In another example, software 1420 may reside, completely or partially, within processor 1404.

Computer system 1400 may also include an input device 1432. In one example, a user of computer system 1400 may enter commands and/or other information into computer system 1400 via input device 1432. Examples of an input device 1432 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1432 may be interfaced to bus 1412 via any of a variety of interfaces (not shown) including, but not limited to, a serial interface, a parallel interface, a game port, a USB interface, a FIREWIRE interface, a direct interface to bus 1412, and any combinations thereof. Input device 1432 may include a touch screen interface that may be a part of or separate from display 1436, discussed further below. Input device 1432 may be utilized as a user selection device for selecting one or more graphical representations in a graphical interface as described above.

A user may also input commands and/or other information to computer system 1400 via storage device 1424 (e.g., a removable disk drive, a flash drive, etc.) and/or network interface device 1440. A network interface device, such as network interface device 1440, may be utilized for connecting computer system 1400 to one or more of a variety of networks, such as network 1444, and one or more remote devices 1448 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1444, may employ a wired and/or a wireless mode of communication. In general, any network topology may be used. Information (e.g., data, software 1420, etc.) may be communicated to and/or from computer system 1400 via network interface device 1440.

Computer system 1400 may further include a video display adapter 1452 for communicating a displayable image to a display device, such as display device 1436. Examples of a display device include, but are not limited to, a liquid crystal display (LCD), a cathode ray tube (CRT), a plasma display, a light emitting diode (LED) display, and any combinations thereof. Display adapter 1452 and display device 1436 may be utilized in combination with processor 1404 to provide graphical representations of aspects of the present disclosure. In addition to a display device, computer system 1400 may include one or more other peripheral output devices including, but not limited to, an audio speaker, a printer, and any combinations thereof. Such peripheral output devices may be connected to bus 1412 via a peripheral interface 1456. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for prioritizing comprehensive prognoses and generating a treatment instruction set, the system comprising:
a computing device, wherein the computing device is designed and configured to:
receive at least a user biological marker;
generate a classification machine-learning model using machine-learning training data wherein the machine-learning training data contains a plurality of data entries containing biological markers as inputs correlated to associated diagnostics as outputs;
determine a diagnostic using the at least a user biological marker, the classification machine-learning model, and the machine-learning training data;
rank the diagnostic, wherein ranking further comprises using a statistical machine-learning process to determine a figure of merit of the diagnostic matching the user biological marker; and
select a prognosis as a function of the figure of merit, wherein selecting the prognosis further comprises:
generating at least a treatment;
performing a simulation machine-learning process, wherein the simulation machine-learning process generates an output containing a prognosis using the at least a treatment as an input;
determining a rank for the prognosis; and
providing an instruction set that results in an optimal prognosis;
display, using a graphical user interface, the instruction set.

2. The system of claim 1, wherein computing device is further configured to:
collect a pattern of biological marker data from a wearable device to generate the at least a user biological marker;
classify, using the classification machine-learning model, the at least a user biological marker data to a diagnostic profile as a function of the pattern of biological marker data.

3. The system of claim 1, wherein generating the at least a treatment further comprises:
retrieving a treatment using a query within a database for a treatment, wherein querying is performed as a function of a user preference;
determining a relevancy rank for the treatment in addressing the diagnostic, wherein the relevancy rank relates to the ability of the treatment to result in an optimal prognosis; and.
providing an instruction set for the treatment, wherein the instruction set is a series of instructions a user may perform to address the diagnostic.

4. The system of claim 3, wherein the treatment further comprises a supervised machine-learning model.

5. The system of claim 1, wherein generating the at least a treatment further comprises ranking a plurality of instructions contained within the instruction set using a ranking machine-learning process, wherein the ranking machine-learning process ranks the plurality of instructions as a function of a chronological ordering that results in an optimal prognosis for the at least a treatment.

6. The system of claim 1, wherein performing the simulation machine-learning process further comprises:
sampling at least a parameter in the biological marker associated with implementing an instruction set, wherein sampling perturbs the at least a parameter in the biological marker;
determining an effect of at least a simulated parameter in the biological marker, wherein determining further comprises calculating how the at least a parameter in the biological marker may change as a function of the at least a simulated parameter in the biological marker; and
identifying at least a simulated parameter in the biological marker that results in a ranked treatment containing an optimal prognosis from implementing the instruction set.

7. The system of claim 6, wherein the computing device is further configured to modify the instruction set to reflect the at least a simulated parameter.

8. The system of claim 1, wherein ranking the diagnostic further comprises ranking a plurality of diagnostics as a function of a ranking machine-learning process and a figure of merit of each diagnostic.

9. The system of claim 1, wherein the computing device is further configured to:
direct the user to update user data as a function of the instruction set, wherein directing further comprises:
displaying a notification to remind the user to follow-up;
calculating a cooperation rank, wherein the cooperation rank relates a user rank with a level of cooperation and a completion of the instruction set; and
providing, via the user device, the cooperation rank.

10. The system of claim 1, wherein the computing device is further configured to:
receive updated biological marker data, where updated biological marker data is more recent in time than the at least a user biological marker
compute, using the classification machine-learning model and the updated biological marker data, a numerical difference between the figure of merit and an updated figure of merit; and
determine a prognosis state as a function of the numerical difference.

11. A method of prioritizing comprehensive prognoses and generating a treatment instruction set, the method comprising:
receiving, by a computing device, at least a user biological marker;
generating, by the computing device, a classification machine-learning model using machine-learning training data wherein the machine-learning training data contains a plurality of data entries containing biological markers as inputs correlated to associated diagnostics as outputs;
determining, by the computing device, a diagnostic using the at least a user biological marker, the classification machine-learning model, and the machine-learning training data;
ranking, by the computing device, the diagnostic, wherein ranking further comprises using a statistical machine-learning process to determine a figure of merit of the diagnostic matching the user biological marker; and
selecting, by the computing device, a prognosis as a function of the figure of merit, wherein selecting the prognosis further comprises:
generating at least a treatment;
performing a simulation machine-learning process, wherein the simulation machine-learning process generates an output containing a prognosis using the at least a treatment as an input;
determining a rank for the prognosis; and
providing an instruction set that results in an optimal prognosis;
displaying, by the computing device, using a graphical user interface, the instruction set.

12. The method of claim 11, wherein the at least a user biological marker further comprises:
collecting a pattern of biological marker data from a wearable device to generate the at least a user biological marker;
classifying, using the classification machine-learning model, the at least a user biological marker data to a diagnostic profile as a function of the pattern of biological marker data.

13. The method of claim 11, wherein generating the at least a treatment further comprises:
retrieving a treatment using a query within a database for a treatment, wherein querying is performed as a function of a user preference;
determining a relevancy rank for the treatment in addressing the diagnostic, wherein the relevancy rank relates to the ability of the treatment to result in an optimal prognosis; and.
providing an instruction set for the treatment, wherein the instruction set is a series of instructions a user may perform to address the diagnostic.

14. The method of claim 13, wherein the treatment further comprises a supervised machine-learning model.

15. The method of claim 11, wherein generating the at least a treatment further comprises ranking a plurality of instructions contained within the instruction set using a ranking machine-learning process, wherein the ranking machine-learning process ranks the plurality of instructions as a function of a chronological ordering that results in in an optimal prognosis for the at least a treatment.

16. The method of claim 11, wherein performing the simulation machine-learning process further comprises:
sampling at least a parameter in the biological marker associated with implementing an instruction set, wherein sampling perturbs the at least a parameter in the biological marker;
determining an effect of at least a simulated parameter in the biological marker, wherein determining further comprises calculating how at least a parameter in the biological marker may change as a function of the at least a simulated parameter in the biological marker; and
identifying at least a simulated parameter in the biological marker that results in a ranked treatment containing an optimal prognosis from implementing the instruction set.

17. The method of claim 16, further comprising modifying the instruction set to reflect the at least a simulated parameter.

18. The method of claim 11, wherein ranking the diagnostic further comprises ranking a plurality of diagnostics as a function of a ranking machine-learning process and a figure of merit of each diagnostic.

19. The method of claim 11 further comprising:
directing the user to update user data as a function of the instruction set, wherein directing further comprises:
displaying a notification to remind the user to follow-up;
calculating a cooperation rank, wherein the cooperation rank relates a user rank with a level of cooperation and a completion of the instruction set; and
providing, via the user device, the cooperation rank.

20. The method of claim 11 further comprising:
Receiving, updated biological marker data, where updated biological marker data is more recent in time than the at least a user biological marker;
computing, using the classification machine-learning model and the updated biological marker data, a numerical difference between the figure of merit and an updated figure of merit; and
determining, a prognosis state as a function of the numerical difference.
